SEQUENCE LISTING (5 sequences)

SEQ ID NO: 1 - DNA, 1874 bp, Homo sapiens
gaattccggcaggcacaaactcatccatccccagttgattggaagaaacaacgatgactcctgggaagacctcattggtgtcactgctactgctgctgagcctggaggccatagtgaaggcaggaatcacaatcccacgaaatccaggatgcccaaattctgaggacaagaacttcccccggactgtgatggtcaacctgaacatccataaccggaataccaataccaatcccaaaaggtcctcagattactacaaccgatccacctcaccttggaatctccaccgcaatgaggaccctgagagatatccctctgtgatctgggaggcaaagtgccgccacttgggctgcatcaacgctgatgggaacgtggactaccacatgaactctgtccccatccagcaagagatcctggtcctgcgcagggagcctccacactgccccaactccttccggctggagaagatactggtgtccgtgggctgcacctgtgtcaccccgattgtccaccatgtggcctaagagctctggggagcccacactccccaaagcagttagactatggagagccgacccagcccctcaggaaccctcatccttcaaagacagcctcatttcggactaaactcattagagttcttaaggcagtttgtccaattaaagcttcagaggtaacacttggccaagatatgagatctgaattacctttccctctttccaagaaggaaggtttgactgagtaccaatttgcttcttgtttacttttttaagggctttaagttatttatgtatttaatatgccctgagataactttggggtataagattccattttaatgaattacctactttattttgtttgtctttttaaagaagataagattctgggcttgggaattttattatttaaaaggtaaaacctgtatttatttgagctatttaaggatctatttatgtttaagtatttagaaaaaggtgaaaaagcactattatcagttctgcctaggtaaatgtaagatagaattaaatggcagtgcaaaatttctgagtctttacaacatacggatatagtatttcctcctctttgtttttaaaagttataacatggctgaaaagaaagattaaacctactttcatatgtattaatttaaattttgcaatttgttgaggttttacaagagatacagcaagtctaactctctgttccattaaacccttataataaaatccttctgtaataataaagtttcaaaagaaaatgtttatttgttctcattaaatgtattttagcaaactcagctcttccctattgggaagagttatgcaaattctcctataagcaaaacaaagcatgtctttgagtaacaatgacctggaaatacccaaaattccaagttctcgatttcacatgccttcaagactgaacaccgactaaggttttcatactattagccaatgctgtagacagaagcattttgataggaatagagcaaataagataatggccctgaggaatggcatgtcattattaaagatcatatggggaaaatgaaaccctccccaaaatacaagaagttctgggaggagacattgtcttcagactacaatgtccagtttctcccctagactcaggcttcctttggagattaaggcccctcagagatcaacagaccaacatttttctcttcctcaagcaacactcctagggcctggcttctgtctgatcaaggcaccacacaacccagaaaggagctgatggggcagaatgaactttaagtatgagaaaagttcagcccaagtaaaataaaaactcaatcacattcaattccagagtagtttcaagtttcacatcgtaaccattttcgcccggaattc

SEQ ID NO: 2 - protein, 155 aa, Homo sapiens
MTPGKTSLVSLLLLLSLEAIVKAGITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 3 - protein, 138 aa, Artificial sequence
  Synthetic peptide
GITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVAHHHHHH

SEQ ID NO: 4 - protein, 137 aa, Artificial sequence
  Synthetic peptide
MIVKAGITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 5 - protein, 149 aa, Artificial sequence
  Synthetic peptide
GITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVANPAFLYKVVDIHHHHHH

(19) United States
(12) Patent Application Publication
(10) Pub. No.: US 2008/0014172 A1
(43) Pub. Date: Jan. 17, 2008

(54) Use of Il-17 in the Treatment of Fertility-Related Disorders

(75) Inventors: Ann M. Clark, Weymouth, MA (US); David Kagan, Brooklyn, MA (US); Stephen S. Palmer, Plympton, MA (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: APPLIED RESEARCH SYSTEMS ARS HOLDING N.V., Curacao (AN)

(21) Appl. No.: 11/596,416

(22) PCT Filed: May 31, 2005
(86) PCT No.: PCT/US05/18952

§ 371 Date: Jul. 30, 2007

Related U.S. Application Data
(60) Provisional application No. 60/575,326, filed on May 28, 2004.

(51) Int. Cl. A61K 38/20; A61P 15/00; C12Q 1/02

(52) U.S. Cl. 424/85.2; 435/29

(57) ABSTRACT

The present invention is directed to methods for ameliorating reproductive disorders. More specifically, the present invention describes methods and compositions for using IL-17 in the treatment of various infertility-related defects.

### BACKGROUND

1. Field of the Invention

The present invention is generally directed to the treatment of infertility. More particularly, the present application is directed to treating infertility using IL-17.

2. Background of the Related Art

It has been estimated that there are in excess of 6 million couples in the United States alone that suffer from infertility. A major factor leading to infertility in women is endometriosis, a disorder which affects about one in five women of reproductive age, and as many as one in two women with fertility problems (Brosens, Am. J. Obstet. Gynecol. 176:263-7, 1997; Child and Tan, Drugs 61:1735, 2001). It is the second most common disease in women and is characterized by the occurrence of endometrial cells outside the womb. The presence of these cells outside of the womb lead to progressive, disabling dysmenorrheal and pelvic pain during and after sexual intercourse as well as around the time of menses. The dysmenorrheal pain includes backache, diarrhea, dizziness, headache and nausea.

The endometrium is the lining mucosa of the inner surface of the uterus. Under normal circumstances, the endometrium is only found in the womb. However, in endometriosis, endometrial tissue is shed through fallopian tubes during menstruation, which is termed “retrograde menstruation,” and implants at various sites in the peritoneal cavity and grows into calli of endometrial masses (referred to herein as endometrial foci or calli). The sites of implantation on the external surfaces of the reproductive organs include the uterus, ovaries, uterosacral ligaments, pelvic peritoneum, rectovaginal septum, cervix, vagina, and the fallopian tubes. In addition, cellular adhesion can also occur in the abdominal cavity on the intestine, bladder, pleura, lymph nodes and the pancreas. The extrauterine endometriotic foci located outside the womb are still influenced by hormones of the female cycle and in response to those hormone levels, bleed and undergo volume changes that can contribute to the pain associated with endometriosis. In addition, when increased cellular adhesion occurs in the ovaries and fallopian tubes of endometriotic women, it can result in mechanical sterility of these women.

The presence of endometrial cells outside the womb can also cause an inflammatory response in endometrial women. The inflammatory response can, in turn, further contribute to the pain associated with endometriosis. The uterine fluids of endometriotic women have been found to have high concentrations of various inflammatory cytokines. One of the several such macrophage secretory products involved in endometriotic inflammatory reaction is Tumor Necrosis Factor (TNF, also known as cachectin; Overton et al., Hum. Reprod. 11, 380-386, 1996). TNF is a pleiotropic cytokine released by activated T cells and macrophages. It is a member of the interferon, interleukin and colony stimulating factor cytokine network. This network of factors plays a key role in the signaling system that leads to the pathogenesis of many infectious and inflammatory diseases. TNF induces a number of proinflammatory changes, including production of other cytokines and adhesion molecules (Fiers, FEBS Lett. 285, 199-212, 1991). Elevated levels of macrophages and TNF-α in the peritoneal cavity are associated with endometriosis. (Hornung et al., Am J Pathol. 2001 June; 158(6):1949-54; Rana et al., Fertil Steril. 1996 May; 65(5):925-30.) Other macrophage secretory products that have been found at elevated concentrations in the peritoneal fluid of women with endometriosis, include RANTES (Hornung et al., J. Clin. Endocrinol. Metab. 82, 1621-1628, 1997), Interleukin-6 (Harada et al., Am. J. Obstet. Gynecol. 176, 593-597, 1997), Interleukin-8 (Arici et al., Mol. Hum. Reprod. 2, 40-45, 1996), Monocyte Chemotactic Protein-1 (Arici, et al., Fertil. Steril. 67, 1065-1072, 1997). Granulocyte/macrophage-colony stimulating factor (GM-CSF) has been found elevated in adenomyotic endometrium and in endometriotic tissues relative to unaffected endometrium from matched biopsies, and been found elevated during the secretory phase of the menstrual cycle (Propst et al., 2002; Sidell et al., 2002; Sharpe-Timms et al., 1994). Immunological changes have been demonstrated in women with endometriosis (Rana et al., Fertil. Steril. 65, 925-930, 1996). In response to TNF-α, endometriotic epithelial cells have been shown to produce MCP-1 and express elevated levels of IL-6, IL-8, and to express elevated levels of N-cadherin, whereas eutopic endometrial cells express lower amounts of MCP-1 and N-cadherin in these conditions. In addition, TNF-a has also been shown to stimulate proliferation of endometrial stromal cells, and the effect is mediated by IL-8. Granulocyte/macrophage-colony stimulating factor (GM-CSF) has been found elevated in adenomyotic endometrium and in endometriotic tissues relative to unaffected endometrium from matched biopsies, and been found elevated during the secretory phase of the menstrual cycle (Propst et al., 2002; Sidell et al., 2002; Sharpe-Timms et al., 1994).

At the same time as playing a significant role in the pathology of endometriosis, it is known that TNF-α is associated with transformation of human endometrium towards an implantation receptive phenotype, i.e., TNF-α is required for the decidualization of stromal endothelial cells to make the stromal cells amenable to receiving an implant. TNF-α promotes production of HB-EGF from human endometrial stromal cells, which is associated with the window of implantation. TNF-α, as well as several cytokines and chemokines have been found elevated in decidualized human uterine stromal cells in vitro (Popovici et al., Endocrinology. 2000 September; 141(9):3510-3, 2000). TNF-α has also been shown to increase prostaglandin production from endometrial epithelial cells co-incubated with human uterine stromal cells obtained during the luteal phase of the cycle, coincident with the window of implantation. Among the cytokines reported to be elevated in endometriosis, IL-6, IL-8, GM-CSF-1 have also been shown to be strongly associated with a receptive endometrium appropriate for embryo implantation. While leukemia inhibitory factor (LIF) and IL-11 have been claimed to have indisputable roles in facilitating implantation combined elevation of TNF-α and LIF levels were found in uterine fluid among patients with failed implantation, whereas women who became pregnant had lower levels of TNF and LIF (Ledee-Bataille et al., Hum Reprod. 17(1):213-8, 2002).

Morphologically, the endometriotic lesions themselves are progressive, appearing initially as clear vesicles, which subsequently become red and then progress to black, fibrotic lesions over a period of few years (MacSween, Muir's Textbook of pathology, 13th ed. (Ed. Whaley K), 1024-1025, 1993). Therefore, initially, the existence of translocated endometrial cells can only be proven microscopically on the peritoneal lining of the pelvis, or even at extra-pelvic sites such as the diaphragm in the upper abdomen (Murphy et al., Fert. and Sterility 46: 522-524 (1986)). However, regular menstrual bleeding at the sites of endometrial foci over time leads to angiogenesis and growth of visible lesions where none had been previously visible. Pain and infertility develop from the bleeding at, and into those sites (Brosens, Am. J. Obstet. Gynecol. 176:263-7, 1997). Symptoms of endometriosis usually subside during pregnancy and lactation, after castration premenopausally, and as the hypoestrogenemia of the peri-menopause develops. Nevertheless, endometriosis is a life-long pathological condition that may be stimulated at any age by unopposed estrogen therapy to produce recurrent symptoms.

Because ectopic endometrial tissue is sensitive to estrogen, the main non-surgical treatments for endometriosis involve blocking estrogen action or synthesis. The use of GnRH agonists, which cause a decrease in gonadotropin secretion by the pituitary and subsequent reduction in ovarian estrogen production, have been approved by the FDA as a six-month course of treatment for endometriosis (Henzl et al., N. Eng. J. Med. 318: 385-9, 1988). Repeated treatment is hindered by the fact that such repeat treatment induces a menopausal-like state and may lead to bone loss and osteoporosis. Further, the symptoms and signs of endometriosis recur in at least 50% of patients within five years after completion of GnRH-agonist treatment (Waller et al., Fertility and Sterility 59:511-515, 1993; Regidor et al., Eur. J. Obstet. Gynecol. Reprod. Biol. 73: 153-60, 1997).

In addition to GnRH agonists, other hormonal therapies have included high dose oral contraceptive pills to mimic pseudopregnancy (using high doses of estrogen and progesterone), and Danazol (an androgenic derivative of ethisterone). These hormonal therapies have some ameliorative effects on the pain, but their extended use is hindered by other side effects. For example, the high dose oral contraceptives stimulate and cause proliferation of endometriotic tissue since it may be unable to respond to progesterone, even at high doses. As such, oral contraceptive pills offer limited relief (Dawood, Int. J. Gynaecol. Obstet. 40 (Suppl.), S29-42, 1993). Progesterones tend to cause irregular bleeding along with depression, weight gain, and fluid retention. Danazol, suppresses endometriosis evoking various responses, including the reduction of soluble TNFα, Interleukin-1β and CD8 levels in serum (Matalliotakis, Int. J. Fertil. Womens. Med. 42, 211-214, 1997; Mori, Am. J. Reprod. Immunol. 24, 45-50, 1990), the inhibition of de novo steroidogenesis and displacement of estradiol from its receptor. Danazol can improve symptoms in approximately 66-100% of the patients suffering from pain, but after less than 4 years there is recurrence of the disease in 40%-500% of cases. In addition, Danazol therapy leads to weight gain and androgenic side effects, which can cause up to 80% of patients to abandon this therapy (Barbieri, N. Engl. J. Med. 318, 512-514, 1988).

The alternative to hormonal therapy of endometriosis is surgical intervention. In cases where the endometriosis is severe, removal of the uterus, tubes and ovaries is indicated. If the disease is less marked, then a less invasive surgical resection of the endometrial polyps may be performed, however, even limited surgical treatment leads to a significant decrease in fertility. Pregnancy rates following surgery generally range between 35% and 65%, leaving many patients requiring ART to achieve normal fecundity (Koninckx and Martin, Curr. Opin. Obstet. Gynecol. 6, 231.241, 1994). Furthermore, even after laparotomy and resection of endometriotic lesions, up to 40% of patients experience recurrent endometriosis requiring further surgical intervention within 5 years. Indeed, recurrence of endometriosis continues even in those patients that receive more drastic surgical intervention. Therefore, surgery alone is insufficient to cure the disease (Revelli et al., Obstet. Gynecol. Surv. 50, 747-754, 1995).

Thus, even though current therapies for the treatment of infertility in general may have some ameliorative effect, those therapies are inadequate for long-term treatment of a disorder that is recurrent and life long. Therefore, there is a need for further therapies for the treatment of endometriosis and other infertility disorders.

### SUMMARY OF THE INVENTION

The present invention provides methods for ameliorating reproductive disorders using IL-17. More specifically, in certain embodiments there are provided methods for the treatment of a fertility-related disorder in a mammal comprising administering to the mammal a composition comprising IL-17 in an amount effective to alleviate the symptoms of the fertility related disorder. In particularly preferred embodiments, the methods of the invention are useful in the treatment of endometriosis. Contemplated herein are methods for treating endometriosis in a mammal comprising administering to the mammal a composition comprising IL-17 in an amount effective to alleviate at least one symptom of endometriosis. In the endometriosis treatment methods contemplated herein, the IL-17 is administered in an amount effective to inhibit the cellular adhesion of sloughed endometrial tissue to surrounding tissue. In certain embodiments, the effect of the administration of IL-17 is to reduce the number of endometrial lesions in the mammal.

The treatment methods may entail administration of IL-17 alone or in combination with another agent useful in the treatment of a fertility disorder such as endometriosis. In certain embodiments, the methods may further comprise administering an agent that inhibits the production, activity or expression of TNFα or its receptor. For example, such methods may comprise administration of an anti-TNF antibody or a fragment thereof. In other embodiments, the agent may be an anti-inflammatory agent such as a non-steroidal anti-inflammatory agent.

In particular embodiments, combination therapies are contemplated wherein the IL-17 is administered in combination with an agent that treats endometriosis. For example, the composition comprising IL-17 may be administered in combination with surgical resection of endometrial tissue. Typically, the composition comprising IL-17 may be administered before, after or during the surgical resection. The methods of the present invention may further comprise administering hormone therapy, such as for example, administering a gonadotropin releasing-hormone (GnRH) agonist. The GnRH agonist may be selected from the group consisting of nafarelin acetate, leuprolide acetate, goserelin acetate, and buserelin acetate. The composition comprising IL-17 is administered concurrently with the GnRH agonist or is administered upon cessation of GnRH agonist.

The hormone therapeutic methods may comprise administering an estrogen agent with a progestin agent to the woman on a daily basis for a period of time sufficient to prevent the symptoms of endometriosis. The estrogen agent is administered at a level that is biologically equivalent to about 5 to about 35 micrograms of ethinyl estradiol and the progestin agent is biologically equivalent to about 0.2 to about 1.5 milligrams of norethindrone acetate. The estrogen agent and the progestin agent are coadministered transdermally or orally. The oral administration of estrogen agent and the progestin agent is as a monophasic tablet or capsule.

Another aspect of the present invention is directed to methods of promoting decidualization of endometrial stromal cells in a mammal comprising administering to the mammal a composition comprising IL-17 in an amount effective to render the endometrial stromal cells of said mammal receptive to implantation as compared to cells of said mammal prior to administration of said composition. The promotion of decidualization an be monitored through routine techniques, including for example techniques that monitor a characteristic selected from the group consisting of an increase IGFBP-1 expression, an increase in IL-1β expression, activation COX-2 expression, an increase in intracellular cAMP, decreased a-smooth muscle actin expression, an increase in PGE2 production, an increase in prolactin production, an increase in amount of oocyte implantation, and a swelling of endometrial stromal cells in response to said IL-17 administration. In specific embodiments, the decidualization is effectively increased in a mammal that is suffering from endometriosis and administration of said IL-17 reverses the endometriotic phenotype of the endometrial cells to a phenotype that is receptive to embryo implantation.

Other aspects of the present invention describe methods of screening for an agent that inhibits TNFα-induced endometrial cell adhesion the method comprising:

i) determining the binding of endometrial cells in culture with a solid support in the presence of TNFα;

ii) determining the binding of endometrial cells in culture with a solid support in the presence of TNFα in the presence of IL-17;

iii) determining the binding of endometrial cells in culture with a solid support in the presence of TNFα, IL-17 and a candidate agent; and comparing the binding in step (ii) with step (iii), wherein a decrease in binding between step (ii) and step (iii) indicates that the agent is an inhibitor of TNFα-induced endometrial cell adhesion.

The invention also contemplates a composition comprising IL-17 for use in the treatment of infertility. The invention further contemplated the use IL-17 for the manufacture of a medicament useful in the treatment of infertility. Further compositions of the invention IL-17 for use in the treatment of endometriosis. The invention further contemplated the use IL-17 for the manufacture of a medicament useful in the treatment of endometriosis.

Other features and advantages of the invention will become apparent from the following detailed description. It should be understood, however, that the detailed description and the specific examples, while indicating preferred embodiments of the invention, are given by way of illustration only, because various changes and modifications within the spirit and scope of the invention will become apparent to those skilled in the art from this detailed description.

### BRIEF DESCRIPTION OF THE FIGURES

The following drawings form part of the present specification and are included to further illustrate aspects of the present invention. The invention may be better understood by reference to the drawings in combination with the detailed description of the specific embodiments presented herein.

FIG. 1. Binding of fluorescent fibronectin-coated beads to BEND cells that have been treated with TNFα or TNFα (15 ng/ml)+TNFα binding protein (TBP).

FIG. 2. Dose-response of IL-17-6His.

FIG. 3. Dose-response of MET-IVKA-IL-17.

FIG. 4. Dose-response of IL-17-ATT-6His.

FIG. 5. Dose-response of two separate IL-17 preparations compared to the positive control TBP.

FIG. 6. Effect of IL-17 and TNF-a on GM-CSF production from 12Z endometriotic epithelial cells.

FIG. 7. Effect of IL-17 and TNF-a on IL-6 production from 12Z endometriotic epithelial cells.

FIG. 8. Effect of IL-17 and TNF-a on IL-8 production from 12Z endometriotic epithelial cells.

FIG. 9. Effect of IL-17 on TNF-a or IL-1b-stimulated GM-CSF production from human endometrial stromal cells (HuF6 cells).

FIG. 10. Differential effects of IL-17 on TNF-a-stimulated production of IL-6 compared to IL-1b and the combination of IL-1b and IL-17.

FIG. 11. Differential effects of IL-17 on TNF-a-stimulated production of IL-8 compared to IL-1b and the combination of IL-1b and IL-17.

### DESCRIPTION OF THE PREFERRED EMBODIMENTS

There remains a consistent need for new methods for the treatment of infertility-related disorders in the United States as well as the rest of the world. Infertility is a multifactoral condition brought about by any one or more of several physiological factors. One such factor is the syndrome of endometriosis. This disorder is characterized by the adhesion of endometrial tissue other than the lining of the uterus, the natural locus of the endometrial tissue. Endometriosis has attendant physical and psychological pain that is not only debilitating to the health of the woman but it also is a major cause of infertility. The current methods for the treatment of endometriosis are inadequate to overcome the devastating effects of this disease.

The present invention is directed to new methods and compositions for the treatment of endometriosis and other infertility disorders. While much of the discussion presented herein is directed to endometriosis, it should be understood that the methods of the present invention may be used in the treatment of infertility generally. Thus, in certain aspects, the methods of the present invention may be used in the treatment of any infertility disorder, but especially those that manifest as a result of an aberrant or overexpression of TNFα. Such disorders include but are not limited to endometriosis (including manifestations of endometriosis that occur at sites distal to the reproductive organs). The methods of the present invention may be used to alleviate one or more signs or symptoms of endometriosis including the symptoms of adenomyosis and dysmenorrhea as discussed herein throughout.

In certain aspects, the data presented herein show that interleukin 17 (IL-17) causes an inhibition of TNFα-induced binding of fibronectin coated materials to endometrial cells thereby demonstrating that IL-17 inhibits TNFα-associated endometriosis effects. The findings of the present invention also facilitate screening assays for the identification of additional agents that would alter the progression of endometriosis.

In further examples provided herein show a physiologic response of IL-17 in the endometrium associated with the transformation from an endometriotic phenotype to an endometrium compatible with implantation is shown. A conundrum in endometrial biology is that TNF-α can be expressly associated with a disease process such as endometriosis that often results in infertility, but at the same time TNF-a is critical for preparation of endometrium for implantation. A concise summary of current literature suggests that other local modulators affect the response to TNF and direct endometrial fate, whether towards an endometriotic phenotype, or towards a decidualization state. The majority of comparative studies between diseased and unaffected endometrium have focused on stromal cells while little attention has been directed towards changes in endometrial epithelial cells. The objective of the studies conducted here was to first examine effects of IL-17 on epithelial cell responses of both endometriotic and endometrial cells, and second to confirm that changes in endometrial stromal cell function are comparable or compatible with those measured in epithelial cells.

In the further studies provided herein, it was shown that the combination of IL-17 and TNF-alpha induces a dramatic increase in production of GM-CSF by human endometrotic epithelial cells. Further, it is demonstrated herein that a synergistic combination of IL-17 and TNF-α also promotes a biochemical transformation of endometrial stromal cells that resembles events associated with decidualization. In stromal cells, IL-17 and TNF-α cause additive effects on GM-CSF production, and synergistic effects on IL-6 and IL-8 production. Production of GM-CSF from epithelial cells, and production of IL-6 and IL-8 are all published markers of a receptive endometrium compatible with implantation. These results suggest that IL-17 in combination with locally produced TNF-α promotes transformation of human endometrial cells that are more compatible with implantation. These results further indicate that IL-17 can improve implantation rates in women with endometriosis and in women with recurrent implantation failure.

It is evident from the above discussion that TNF-a has a dual role in modulating the function of endometrial tissue. On the one hand TNF-a is clearly linked with an endometriosis phenotype. Conversely, it is clear that the TNF-a function also is needed in order to make endometrial stromal cells receptive to embryo implantation, It is demonstrated herein that IL-17 is able to modulate the function of TNFa such that in endometriosis IL-17 can be employed to abrogate or inhibit the endometriosis phenotype whereas in other circumstances IL-17 can be used to render the endometrium more amenable to implantation (i.e., more receptive to implantation). Methods and compositions for exploiting these findings are described in further detail herein below.

Effects of IL-17 on TNFα Action

As described in further detail in the examples herein below, IL-17 was identified as a positive protein in the inhibition of TNFα-induced binding of a solid support to endometrial cells. Thus; in this context, the IL-17 is acting as an inhibitor or antagonist of deleterious effects of TNFα. As discussed below, this finding alone is surprising and unexpected because previously it has been shown that IL-17 activates TNFα release (see U.S. Pat. No. 6,569,645 especially Examples 28 and 30). The present section provides a description of IL-17 and TNFα and methods and compositions for making IL-17 compositions for the various methods described herein.

IL-17 is a cytokine that was described by Rouvier et al. in 1993 as a cDNA termed CTLA-8 (J. Immunol. 150:5445; 1993). Cytokines are hormone-like molecules that regulate various aspects of an immune or inflammatory response. Cytokines exert their effects by specifically binding receptors present on cells, and transducing a signal to the cells. IL-17 has been described as a proinflammatory cytokine that stimulates the expression of other cytokines. In particular, numerous reports have shown that IL-17 stimulates the production and expression of proinflammatory cytokines, IL-1β and TNF-α (Jovanovic et al., J. Immunol. 160:3513-3521, 1998; U.S. Pat. No. 6,569,645), as well as to induce in vivo granulopoiesis (Fossiez et al., J. Exp. Med. 183:2593-2603, 1996 (see also associated GenBank Acc. No. Z58820); Schwarzenberger et al., J. Immunol. 161:6383-6389, 1998; Schwarzenberger et al., J. Immunol. 164:4783-4789, 2000) and induce and CXC chemokines (Laan et al., J. Immunol. 162:2347-2352, 1998).

The present application, for the first time describes the use of IL-17 to inhibit the action of TNFα on cell binding of fibronectin, and in particular, endometrial cells. Given this finding, compositions comprising IL-17 or an IL-17 agonist or stimulator will be useful in inhibiting the effects of TNFα, particularly in reproductive diseases.

The sequence of IL-17 protein and nucleic acids for encoding such proteins are well known to those of skill in the art. For example, Genbank Acc. No. AY460616 provides the complete cds sequence of homo sapiens IL-17 gene; Genbank Acc. No. BD265544; Genbank Acc. No. BD265545; Genbank Acc. No. BD265546; Genbank Acc. No. BD265547; Genbank Acc. No. BD265548; Genbank Acc. No. BD265549; Genbank Acc. No. BD265550; Genbank Acc. No. BD265551; Genbank Acc. No. BD265552; Genbank Acc. No. BD265553; Genbank Acc. No. BD265554; Genbank Acc. No. BD265555; Genbank Acc. No. BD265556; Genbank Acc. No. BD265557, Genbank Acc. No. BD265558, Genbank Acc. No. BD265559, provide IL-17-associated mammalian cytokine sequences and polynucleotides encoding the same that are described in further detail in Japanese Patent No, 2002534122 (incorporated herein by reference). The complete mRNA sequence of human IL-17 also is given at GenBank Acc. No. U32659 and is further described in Yao et al., (J. Immunol. 155 (12), 5483-5486 (1995). The IL-17 sequence from U32659 is reproduced herein as SEQ ID NO:1 (nucleic acid sequence) and SEQ ID NO:2 (protein sequence). However, it should be understood that, as those of skill in the art are aware of the sequence of these molecules, any IL-17 protein or gene sequence variant may be used as long as it has the properties of an IL-17 cytokine.

In addition to the numerous literature references describing the sequence of IL-17, incorporated herein by reference in their entirety are the teachings provided in U.S. Pat. No. 6,043,344, which describes human, rat and herpesvirus herpes IL-17 proteins and nucleic acid compositions. SEQ ID NO:1 and SEQ ID NO:2 from U.S. Pat. No. 6,043,344 are particularly incorporated herein by reference as are the teaching of methods of making variants of these sequences taught in that patent and the methods of testing the compositions in various assays described therein. Also incorporated herein by reference is U.S. Pat. No. 6,074,849. U.S. Pat. No. 6,569,645 also is incorporated herein by reference as providing a teaching of polypeptides homologous to IL-17 and nucleic acid molecules encoding those polypeptides. Other variants of IL-17 that may be useful in the present application include the IL-17E polypeptides and IL-17E-encoding polynucleotides that are described in U.S. Pat. No. 6,579,520.

In exemplary embodiments, the IL-17 compositions used in the methods described herein have a sequence of SEQ ID NO:3: GITIPRNPGC PNSEDKNFPR TVMVNLNIHN RNTNTNPKRS SDYYNRSTSP WNLHRNEDPE RYPSVIWEAK CRHLGCINAD GNVDYHMNSV PIQQEILVLR REPPHCPNSF RLEKILVSVG CTCVTPIVHH VAHHHHHH; SEQ ID NO:4: MIVKAGITIP RNPGCPNSED KNFPRTVMVN LNIHNRNTNT NPKRSSDYYN RSTSPWNLHR NEDPERYPSV IWEAKCRHLG CINADGNVDY HMNSVPIQQE ILVLRREPPH CPNSFRLEKI LVSVGCTCVT PIVHHVA; or SEQ ID NO:5: GITIPRNPGC PNSEDKNFPR TVMVNLNIHN RNTNTNPKRS SDYYNRSTSP WNLHRNEDPE RYPSVIWEAK CRHLGCINAD GNVDYHMNSV PIQQEILVLR REPPHCPNSF RLEKILVSVG CTCVTPIVHH VANPAFLYKV VDIHHHHHH. However, these are merely exemplary sequences and those of skill in the art will be able to employ other variants in the methods described herein.

It is particularly contemplated that conservative substitution of amino acid residues of SEQ ID NO:2; 3, 4, or 5 may be produced that nonetheless retain the three-dimensional conformation structure of these proteins and/or retain the functional cytokine activity of these proteins. In preferred embodiments, the activity to be retained should be one which inhibits, reduces, antagonizes, or otherwise decreases the TNFα-mediated binding of a fibronectin-coated solid support to endometrial cells in an assay such as the BEND assay described in the examples herein below. Both shed human endometrial cells (Koks et al., Mo. Hum. Reprod. 6:17-177, 2000) and cells from human peritoneal mesothelium, the cell type that endometrial cells bind to during endometriosis, express integrin receptors for fibronectin (Witz et al., Fertil. Steril. 74:579-584, 2000), a ligand involved in cellular adhesion. Blocking the integrins with a specific antibody can reduce binding of human endometrial cells to fibronectin (Koks et al.,), and treatment of human endometriotic cells with TNFα significantly increases adhesion to fibronectin (Sillem et al., Eur. J. Obstet. Gynecol. Reprod. Biol. 87:123-127, 1999). In the BEND assay, bovine endometrial cells are treated with TNFα to increase binding of fibronectin-coated fluorescent beads as a measure of cellular adhesion. The fluorescence of the beads allow a facile measurement of fibronectin binding. TNFα is used as an inducer of fibronectin binding because of its known presence in the peritoneal fluid of women with endometriosis and its involvement in the disease (Bullimore, Med. Hypotheses 60:84-88, 2003.)

The term “conservative substitution” as used herein denotes the replacement of an amino acid residue by another, biologically similar residue with respect to hydrophobicity, hydrophilicity, cationic charge, anionic charge, shape, polarity and the like. Examples of conservative substitutions include the substitution of one hydrophobic residue such as isoleucine, valine, leucine, alanine, cysteine, glycine, phenylalanine, proline, tryptophan, tyrosine, norleucine or methionine for another, or the substitution of one polar residue for another, such as the substitution of arginine for lysine, glutamic acid for aspartic acid, or glutamine for asparagine, and the like. Neutral hydrophilic amino acids which can be substituted for one another include asparagine, glutamine, serine and threonine. The term “conservative substitution” also includes the use of a substituted or modified amino acid in place of an unsubstituted parent amino acid provided that antibodies raised to the substituted polypeptide also immunoreact with the unsubstituted polypeptide. By “substituted” or “modified” the present invention includes those amino acids that have been altered or modified from naturally occurring amino acids.

Therefore, it should be understood that in the context of the present invention, a conservative substitution is recognized in the art as a substitution of one amino acid for another amino acid that has similar properties. Exemplary conservative substitutions are set out in e.g., Alternatively, conservative amino acids can be grouped as described in Lehninger, [Biochemistry, Second Edition; Worth Publishers, Inc. NY:N.Y. (1975), pp. 71-77]. Those of skill in the art are aware of numerous tables that indicate specific conservative substitutions. One exemplary such table is provided below:

Table of Exemplary Conservative Substitutions

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met (M) | Leu, Phe, Ile |
| Phe (F) | Leu, Val, Ile, Ala |
| Pro (P) | Gly |
| Ser (S) | Thr |
| Thr (T) | Ser |
| Trp (W) | Tyr |
| Tyr (Y) | Trp, Phe, Thr, Ser |
| Val (V) | Ile, Leu, Met, Phe, Ala |

Any conservative variant of any of the sequences of SEQ ID NO: 2, 3, 4 or 5 will be a useful protein for the present invention as long as that protein or a fragment thereof retains its property of being an inhibitor of TNFα-mediated binding of endometrial cells either in vitro or in vivo. Such activities may be readily assessed as described herein below.

In addition to the basic amino acid structure of the peptides, it is contemplated that the IL-17 derived proteins/peptides may be modified to enhance their uptake, circulation, and/or other modifications to render compositions containing such proteins/peptides more therapeutically effective. For example, it has been discovered herein that IL-17 proteins have a beneficial effect on inhibiting binding of a given support to endometrial cells. As such, it is contemplated that IL-17-based compositions will be used in inhibiting the cell adhesion of endometrial cells/tissue at extrauterine sites. Therefore, any modification that allows the targeting/uptake of the IL-17 peptides/proteins to any site where such endometrial cells have or are likely to attach will be useful.

In some examples, it will be desirable to prevent the degradation of the IL-17 proteins/peptides in order to prolong the effects of the proteins. The use of non-hydrolyzable peptide bonds, which are known in the art, along with procedures for synthesis of peptides containing such bonds may be used to make IL-17 proteins that are more resistant to degradation than native IL-17 proteins that do not contain such bonds. Non-hydrolyzable bonds include —[CH2NH]— reduced amide peptide bonds, —[COCH2]— ketomethylene peptide bonds, —[CH(CN)NH]—(cyanomethylene)amino peptide bonds, —[CH2 CH(OH)]—hydroxyethylene peptide bonds, —[CH2O]-peptide bonds, and —[CH2 S]-thiomethylene peptide bonds (see e.g., U.S. Pat. No. 6,172,043).

The IL-17 proteins useful in the invention can be linear, or maybe circular or cyclized by natural or synthetic means. Disulfide bonds between cysteine residues may cyclize a peptide sequence. Bifunctional reagents can be used to provide a linkage between two or more amino acids of a peptide. Other methods for cyclization of peptides, such as those described by Anwer et al. (Int. J. Pep. Protein Res. 36:392-399, 1990) and Rivera—Baeza et al. (Neuropeptides 30:327-333, 1996) are also known in the art and may be used to produce IL-17 compositions that may have a prolonged effect.

Rational drug design may be used to produce structural analogs of the presently known biologically active IL-17-derived proteins. By creating such analogs, it is possible to fashion related proteins which are more active or stable than the natural molecules which have different susceptibility to alteration or which may affect the function of various other molecules. In one approach, one would generate a three-dimensional structure for IL-17-derived protein of interest or a fragment thereof e.g., this can be accomplished by x-ray crystallography, computer modeling or by a combination of both approaches. An alternative approach, “alanine scan,” involves the random replacement of residues throughout molecule with alanine, and the resulting affect on function determined.

Thus, one may design drugs which have improved IL-17-derived protein activity or which act as stimulators or agonists IL-17. By virtue of the availability of cloned IL-17 sequences, sufficient amounts of various IL-17-derived proteins can be produced to perform crystallographic studies. In addition, knowledge of the polypeptide sequences permits computer employed predictions of structure-function relationships.

Furthermore, nonpeptide analogs of IL-17-derived proteins which provide a stabilized structure or lessened biodegradation, are also contemplated. Peptide mimetic analogs can be prepared based on a known protein sequence by replacing one or more amino acid residues of the protein of interest by nonpeptide moieties. Preferably, the nonpeptide moieties permit the peptide to retain its natural confirmation, or stabilize a preferred, e.g., bioactive, confirmation. One example of methods for preparation of nonpeptide mimetic analogs from peptides is described in Nachman et al., Regul. Pept. 57:359-370 (1995). Peptide as used herein embraces all of the foregoing.

In specific embodiments, it is contemplated that IL-17-derived proteins used in the therapeutic methods of the present invention may be modified in order to improve their therapeutic efficacy. Such modification of therapeutic compounds may be used to decrease toxicity, increase circulatory time, or modify biodistribution. For example, the toxicity of potentially important therapeutic compounds can be decreased significantly by combination with a variety of drug carrier vehicles that modify biodistribution. The IL-17-derived proteins used herein are intended to exert a therapeutic effect on endometrial tissue/cells or surrounding tissue in order to inhibit the action of TNFα at such sites and produce a beneficial inhibition of endometrial cell adhesion at that site. As such, any modification that allows the peptide to be taken up and have an effect at any location where endometrial cells have adhered will be useful. As discussed herein, such sites include e.g., the ovaries, uterosacral ligaments, pelvic peritoneum, rectovaginal septum, cervix, vagina, the fallopian tubes, the vulva and extraovarian sites of endometriosis cellular adhesion in the abdominal cavity such as the intestine, the lungs, bladder, skin, pleura, lymph nodes and the pancreas.

A strategy for improving drug viability is the utilization of water-soluble polymers. Various water-soluble polymers have been shown to modify biodistribution, improve the mode of cellular uptake, change the permeability through physiological barriers; and modify the rate of clearance from the body. (Greenwald et al., Crit Rev Therap Drug Carrier Syst. 2000; 17:101-161; Kopecek et al., J Controlled Release, 74:147-158, 2001). To achieve either a targeting or sustained-release effect, water-soluble polymers have been synthesized that contain drug moieties as terminal groups, as part of the backbone, or as pendent groups on the polymer chain.

Polyethylene glycol (PEG), has been widely used as a drug carrier, given its high degree of biocompatibility and ease of modification. Harris et al., Clin Pharmacokinet. 2001; 40(7):539-51 Attachment to various drugs, proteins, and liposomes has been shown to improve residence time and decrease toxicity. (Greenwald et al., Crit Rev Therap Drug Carrier Syst. 2000; 17:101-161; Zalipsky et al., Bioconjug Chem. 1997; 8:111-118). PEG can be coupled to active agents through the hydroxyl groups at the ends of the chain and via other chemical methods; however, PEG itself is limited to at most two active agents per molecule. In a different approach, copolymers of PEG and amino acids were explored as novel biomaterials which would retain the biocompatibility properties of PEG, but which would have the added advantage of numerous attachment points per molecule (providing greater drug loading), and which could be synthetically designed to suit a variety of applications (Nathan et al., Macromolecules. 1992; 25:4476-4484; Nathan et al., Bioconj Chem. 1993; 4:54-62).

Those of skill in the art are aware of PEGylation techniques for the effective modification of drugs. For example, drug delivery polymers that consists of alternating polymers of PEG and tri-functional monomers such as lysine have been used by VectraMed (Plainsboro, N.J.). The PEG chains (typically 2000 daltons or less) are linked to the a- and e-amino groups of lysine through stable urethane linkages. Such copolymers retain the desirable properties of PEG, while providing reactive pendent groups (the carboxylic acid groups of lysine) at strictly controlled and predetermined intervals along the polymer chain. The reactive pendent groups can be used for derivatization, cross-linking, or conjugation with other molecules. These polymers are useful in producing stable, long-circulating pro-drugs by varying the molecular weight of the polymer, the molecular weight of the PEG segments, and the cleavable linkage between the drug and the polymer. The molecular weight of the PEG segments affects the spacing of the drug/linking group complex and the amount of drug per molecular weight of conjugate (smaller PEG segments provides greater drug loading). In general, increasing the overall molecular weight of the block co-polymer conjugate will increase the circulatory half-life of the conjugate. Nevertheless, the conjugate must either be readily degradable or have a molecular weight below the threshold-limiting glomular filtration (e.g., less than 45 kDa). Thus, PEgylated proteins in the range of between 20 and 35 kDa in molecular weight will be useful.

In addition, to the polymer backbone being important in maintaining circulatory half-life, and biodistribution, linkers may be used to maintain the therapeutic agent in a pro-drug form until released from the backbone polymer by a specific trigger, typically enzyme activity in the targeted tissue. For example, this type of tissue activated drug delivery is particularly useful where delivery to a specific site of biodistribution is required and the therapeutic agent is released at or near the site of pathology. Linking group libraries for use in activated drug delivery are known to those of skill in the art and may be based on enzyme kinetics, prevalence of active enzyme, and cleavage specificity of the selected disease-specific enzymes (see e.g., technologies of established by VectraMed, Plainsboro, N.J.). Such linkers may be used in modifying the IL-17-derived proteins described herein for therapeutic delivery.

Methods of Making Proteins

The present invention provides proteins and peptides for use in medicaments for the treatment of infertility. Such proteins or peptides may be produced by conventional automated peptide synthesis methods or by recombinant expression. General principles for designing and making proteins are well known to those of skill in the art.

A. Automated Solid-Phase Peptide Synthesis

The IL-17 proteins or fragments thereof can be synthesized in solution or on a solid support in accordance with conventional techniques. Various automatic synthesizers are commercially available and can be used in accordance with known protocols. See, for example, Stewart and Young, Solid Phase Peptide Synthesis, 2d. ed., Pierce Chemical Co., (11984); Tam et al., J. Am. Chem. Soc., 105:6442, (1983); Merrifield, Science, 232: 341-347, (1986); and Barany and Merrifield, The Peptides, Gross and Meienhofer, eds, Academic Press, New York, 1-284, (1979), each incorporated herein by reference. The proteins can be readily synthesized and then screened in IL-17 receptor binding/activity assays to determine whether the proteins produced possess IL-17-like activity as an initial screen. The proteins/fragments also may be tested in an exemplary endometriosis assay such as the BEND assay described in the examples to assess whether the proteins have a beneficial effect of inhibiting the action of TNFα-induced cell adhesion of endometrial cells. Any IL-17-derived protein that has at least some effect on inhibiting this action of TNFα will be considered useful.

The proteins or fragments thereof may be synthesized by solid-phase technology employing an exemplary peptide synthesizer such as a Model 433A from Applied Biosystems Inc. The purity of any given protein; generated through automated peptide synthesis or through recombinant methods may be determined using reverse phase HPLC analysis. Chemical authenticity of each peptide may be established by any method well known to those of skill in the art. In preferred embodiments, the authenticity may be established by mass spectrometry. Additionally, the peptides may be quantified using amino acid analysis in which microwave hydrolyses are conducted. Such analyses may use a microwave oven such as the CEM Corporation's MDS 2000 microwave oven. The peptide (approximately 2 μg protein) is contacted with e.g., 6 N HCl (Pierce Constant Boiling e.g., about 4 ml) with approximately 0.5% (volume to volume) phenol (Mallinckrodt). Prior to the hydrolysis, the samples are alternately evacuated and flushed with N₂. The protein hydrolysis is conducted using a two-stage process. During the first stage, the peptides are subjected to a reaction temperature of about 100° C. and held that temperature for 1 minute. Immediately after this step, the temperature is increased to 150° C. and held at that temperature for about 25 minutes. After cooling, the samples are dried and amino acid from the hydrolysed peptides samples are derivatized using 6-aminoquinolyl-N-hydroxysuccinimidyl carbamate to yield stable ureas that fluoresce at 395 nm (Waters AccQ Tag Chemistry Package). The samples may be analyzed by reverse phase HPLC and quantification may be achieved using an enhanced integrator.

B. Recombinant Protein Production

As an alternative to automated peptide synthesis, recombinant DNA technology may be employed wherein a nucleotide sequence which encodes a protein of choice is inserted into an expression vector, transformed or transfected into an appropriate host cell and cultivated under conditions suitable for expression as described herein below. Recombinant methods are especially preferred for producing longer polypeptides.

A variety of expression vector/host systems may be utilized to contain and express the peptide or protein coding sequence. These include but are not limited to microorganisms such as bacteria transformed with recombinant bacteriophage, plasmid or cosmid DNA expression vectors; yeast transformed with yeast expression vectors (Giga-Hama et al., Biotechnol Appl Biochem., 30 (Pt 3):235-44, 1999); insect cell systems infected with virus expression vectors (e.g., baculovirus, see Ghosh et al., Mol Ther. 6(1):5-11, 2002); plant cell systems transfected with virus expression vectors (e.g., cauliflower mosaic virus, CaMV; tobacco mosaic virus, TMV) or transformed with bacterial expression vectors (e.g., Ti or pBR322 plasmid; see e.g., Babe et al., Biotechnol Genet Eng Rev.; 17:213-52, 2000); or animal cell systems. Those of skill in the art are aware of various techniques for optimizing mammalian expression of proteins, see e.g., Kaufman, Mol. Biotechnol. 16(2):151-60, 2000; Colosimo et al., Biotechniques, 29(2):314-8, 2000.

Mammalian cells that are useful in recombinant protein productions include but are not limited to VERO cells, HeLa cells, Chinese hamster ovary (CHO) cell lines, COS cells (such as COS-7), W138, BHK, HepG2, 3T3, RIN, MDCK, A549, PC12, K562 and 293 cells. Exemplary protocols for the recombinant expression of the peptide substrates or fusion polypeptides in bacteria, yeast and other invertebrates are known to those of skill in the art and a briefly described herein below. U.S. Pat. No. 6,569,645; U.S. Pat. No. 6,043,344; U.S. Pat. No. 6,074,849; and U.S. Pat. No. 6,579,520 provide specific examples for the recombinant production of IL-17 related proteins and these patents are expressly incorporated herein by reference for those teachings.

In general, expression vectors for use in prokaryotic hosts comprise one or more phenotypic selectable marker genes. Such genes generally encode, e.g., a protein that confers antibiotic resistance or that supplies an auxotrophic requirement. A wide variety of such vectors are readily available from commercial sources. Examples include pSPORT vectors, pGEM vectors (Promega), pPROEX vectors (LTI, Bethesda, Md.), Bluescript vectors (Stratagene), pET vectors (Novagen) and pQE vectors (Qiagen). The DNA sequence encoding the given protein of interest (e.g., IL-17) is amplified by PCR and cloned into such a vector, for example, pGEX-3X (Pharmacia, Piscataway, N.J.) designed to produce a fusion protein comprising glutathione-S-transferase (GST), encoded by the vector, and a protein encoded by a DNA fragment inserted into the vector's cloning site. The primers for the PCR may be generated to include for example, an appropriate cleavage site. Treatment of the recombinant fusion protein with thrombin or factor Xa (Pharmacia, Piscataway, N.J.) is expected to cleave the fusion protein, releasing the polypeptide of interest from the GST portion. The pGEX-3X/IL-17 construct is transformed into E. coli XL-1 Blue cells (Stratagene, La Jolla Calif.), and individual transformants are isolated and grown. Plasmid DNA from individual transformants is purified and partially sequenced using an automated sequencer to confirm the presence of the desired polypeptide-encoding nucleic acid insert in the proper orientation. If the GST/IL-17 fusion protein is produced in bacteria as a soluble protein, it may be purified using the GST Purification Module (Pharmacia Biotech).

Alternatively, the DNA sequence encoding the predicted substrate containing fusion polypeptide may be cloned into a plasmid containing a desired promoter and, optionally, a leader sequence (see, e.g., Better et al., Science, 240:104-143, 1988). The sequence of this construct may be confirmed by automated sequencing. The plasmid is then transformed into E. coli using standard procedures employing CaCl₂ incubation and heat shock treatment of the bacteria (Sambrook et al., supra). The transformed bacteria are grown in LB medium supplemented with carbenicillin, and production of the expressed protein is induced by growth in a suitable medium. If present, the leader sequence will effect secretion of the mature IL-17 peptide or protein and be cleaved during secretion. The secreted recombinant protein is purified from the bacterial culture media using standard protein purification techniques.

Systems for the recombinant protein in yeast host cells are readily commercially available, e.g., the Pichia Expression System (Invitrogen, San Diego, Calif.), following the manufacturer's instructions. Another alternative recombinant production may be achieved using an insect system. Insect systems for protein expression are well known to those of skill in the art. In one such system, Autographa californica nuclear polyhedrosis virus (AcNPV) is used as a vector to express foreign genes in Spodoptera frugiperda cells or in Trichoplusia larvae. The IL-17 protein coding sequence is cloned into a nonessential region of the virus, such as the polyhedrin gene, and placed under control of the polyhedrin promoter. Successful insertion of IL-17 will render the polyhedrin gene inactive and produce recombinant virus lacking coat protein coat. The recombinant viruses are then used to infect S. frugiperda cells or Trichoplusia larvae in which the IL-17 is expressed (Smith et al., J Virol 46: 584, 1983; Engelhard et al., Proc Nat Acad Sci 91: 3224-7, 1994).

Mammalian host systems for the expression of recombinant proteins also are well known to those of skill in the art. Host cell strains may be chosen for a particular ability to process the expressed protein or produce certain post-translation modifications that will be useful in providing protein activity. Such modifications of the polypeptide include, but are not limited to, acetylation, carboxylation, glycosylation, phosphorylation, lipidation and acylation. Post-translational processing which cleaves a “prepro” form of the protein may also be important for correct insertion, folding and/or function. Different host cells such as CHO, HeLa, MDCK, 293, WI38, and the like have specific cellular machinery and characteristic mechanisms for such post-translational activities and may be chosen to ensure the correct modification and processing of the introduced, foreign protein.

It is preferable that the transformed cells are used for long-term, high-yield protein production and as such stable expression is desirable. Once such cells are transformed with vectors that contain selectable markers along with the desired expression cassette, the cells may be allowed to grow for 1-2 days in an enriched media before they are switched to selective media. The selectable marker is designed to confer resistance to selection and its presence allows growth and recovery of cells which successfully express the introduced sequences. Resistant clumps of stably transformed cells can be proliferated using tissue culture techniques appropriate to the cell.

A number of selection systems may be used to recover the cells that have been transformed for recombinant protein production. Such selection systems include, but are not limited to, HSV thymidine kinase, hypoxanthine-guanine phosphoribosyltransferase and adenine phosphoribosyltransferase genes, in tk-, hgprt- or aprt-cells, respectively. Also, anti-metabolite resistance can be used as the basis of selection for dhfr, which confers resistance to methotrexate; gpt, which confers resistance to mycophenolic acid; neo, which confers resistance to the aminoglycoside G418; als which confers resistance to chlorsulfuron; and hygro, which confers resistance to hygromycin. Additional selectable genes that may be useful include trpB, which allows cells to utilize indole in place of tryptophan, or hisD, which allows cells to utilize histinol in place of histidine. Markers that give a visual indication for identification of transformants include anthocyanins, β-glucuronidase and its substrate, GUS, and luciferase and its substrate, luciferin.

C. Expression Constructs for Recombinant Protein Production

In the recombinant production of the IL-17-derived proteins of the invention, it would be necessary to employ vectors comprising polynucleotide molecules for encoding the IL-17-derived proteins. Methods of preparing such vectors as well as producing host cells transformed with such vectors are well known to those skill in the art. The polynucleotide molecules used in such an endeavor may be joined to a vector, which generally includes a selectable marker and an origin of replication, for propagation in a host. These elements of the expression constructs are well known to those of skill in the art. Generally, the expression vectors include DNA encoding the given protein being operably linked to suitable transcriptional or translational regulatory sequences, such as those derived from a mammalian, microbial, viral, or insect genes. Examples of regulatory sequences include transcriptional promoters, operators, or enhancers, mRNA ribosomal binding sites, and appropriate sequences which control transcription and translation.

The terms “expression vector,” “expression construct” or “expression cassette” are used interchangeably throughout this specification and are meant to include any type of genetic construct containing a nucleic acid coding for a gene product in which part or all of the nucleic acid encoding sequence is capable of being transcribed.

The choice of a suitable expression vector for expression of the peptides or polypeptides of the invention will of course depend upon the specific host cell to be used, and is within the skill of the ordinary artisan. Methods for the construction of mammalian expression vectors are disclosed, for example, in Okayama and Berg (Mol. Cell. Biol. 3:280 (1983)); Cosman et al. (Mol. Immunol. 23:935 (1986)); Cosman et al. (Nature 312:768 (1984)); EP-A-0367566; and WO 91/18982. Other considerations for producing expression vectors are detailed in e.g., Makrides et al., Protein Expr Purif., 17(2):183-202; 1999; Kost et al., Curr Opin Biotechnol., 10(5):428-33, 1999. Wurm et al., Curr Opin Biotechnol. 10(2):156-9, 1999 is incorporated herein as teaching factors for consideration in the large-scale transient expression in mammalian cells for recombinant protein production.

The expression construct may further comprise a selectable marker that allows for the detection of the expression of a peptide or polypeptide. Usually the inclusion of a drug selection marker aids in cloning and in the selection of transformants, for example, neomycin, puromycin, hygromycin, DHFR, zeocin and histidinol. Alternatively, enzymes such as herpes simplex virus thymidine kinase (tk) (eukaryotic), β-galactosidase, luciferase, or chloramphenicol acetyltransferase (CAT) (prokaryotic) may be employed. Immunologic markers also can be employed. For example, epitope tags such as the FLAG system (IBI, New Haven, Conn.), HA and the 6×His system (Qiagen, Chatsworth Calif.) may be employed. Additionally, glutathione S-transferase (GST) system (Pharmacia, Piscataway, N.J.), or the maltose binding protein system (NEB, Beverley, Mass.) also may be used. The selectable marker employed is not believed to be important, so long as it is capable of being expressed simultaneously with the nucleic acid encoding a gene product. Further examples of selectable markers are well known to one of skill in the art.

Expression requires that appropriate signals be provided in the vectors, such as enhancers/promoters from both viral and mammalian sources that may be used to drive expression of the nucleic acids of interest in host cells. Usually, the nucleic acid being expressed is under transcriptional control of a promoter. A “promoter” refers to a DNA sequence recognized by the synthetic machinery of the cell, or introduced synthetic machinery, required to initiate the specific transcription of a gene. Nucleotide sequences are operably linked when the regulatory sequence functionally relates to the DNA encoding the protein of interest (i.e., IL-17, a variant and the like). Thus, a promoter nucleotide sequence is operably linked to a given DNA sequence if the promoter nucleotide sequence directs the transcription of the sequence.

Similarly, the phrase “under transcriptional control” means that the promoter is in the correct location and orientation in relation to the nucleic acid to control RNA polymerase initiation and expression of the gene. Any promoter that will drive the expression of the nucleic acid may be used. The particular promoter employed to control the expression of a nucleic acid sequence of interest is not believed to be important, so long as it is capable of directing the expression of the nucleic acid in the targeted cell. Thus, where a human cell is targeted, it is preferable to position the nucleic acid coding region adjacent to and under the control of a promoter that is capable of being expressed in a human cell. Generally speaking, such a promoter might include either a human or viral promoter. Common promoters include, e.g., the human cytomegalovirus (CMV) immediate early gene promoter, the SV40 early promoter, the Rous sarcoma virus long terminal repeat, β-actin, rat insulin promoter, the phosphoglycerol kinase promoter and glyceraldehyde-3-phosphate dehydrogenase promoter, all of which are promoters well known and readily available to those of skill in the art, can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient to produce a recoverable yield of protein of interest. By employing a promoter with well known properties, the level and pattern of expression of the protein of interest following transfection or transformation can be optimized. Inducible promoters also may be used.

Another regulatory element that is used in protein expression is an enhancer. These are genetic elements that increase transcription from a promoter located at a distant position on the same molecule of DNA. Where an expression construct employs a cDNA insert, one will typically desire to include a polyadenylation signal sequence to effect proper polyadenylation of the gene transcript. Any polyadenylation signal sequence recognized by cells of the selected transgenic animal species is suitable for the practice of the invention, such as human or bovine growth hormone and SV40 polyadenylation signals.

Also contemplated as an element of the expression cassette is a terminator. These elements can serve to enhance message levels and to minimize read through from the cassette into other sequences. The termination region which is employed primarily will be one selected for convenience, since termination regions for the applications such as those contemplated by the present invention appear to be relatively interchangeable. The termination region may be native with the transcriptional initiation, may be native to the DNA sequence of interest, or may be derived for another source.

Further, it has been shown that polyhistidylation of nucleic acid molecules is useful in achieving cytosolic delivery of nucleic acids, and that ionic complexes between histidylated polylysine and a pDNA are attractive for developing a nonviral gene delivery system (Midoux et al., Somat Cell Mol. Genet. 2002 November; 27(1-6):27-47).

D. Site-Specific Mutagenesis

Site-specific mutagenesis is another technique useful in the preparation of individual IL-17-derived proteins used in the methods of the invention. This technique employs specific mutagenesis of the underlying DNA (that encodes the amino acid sequence that is targeted for modification). The technique further provides a ready ability to prepare and test sequence variants, incorporating one or more of the foregoing considerations, by introducing one or more nucleotide sequence changes into the DNA. Site-specific mutagenesis allows the production of mutants through the use of specific oligonucleotide sequences that encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

The technique typically employs a bacteriophage vector that exists in both a single stranded and double stranded form. Typical vectors useful in site-directed mutagenesis include vectors such as the M13 phage. These phage vectors are commercially available and their use is generally well known to those skilled in the art. Double stranded plasmids also are routinely employed in site-directed mutagenesis, and eliminates the step of transferring the gene of interest from a phage to a plasmid.

In general, site-directed mutagenesis is performed by first obtaining a single-stranded vector, or melting of two strands of a double stranded vector which includes within its sequence a DNA sequence encoding the desired protein. An oligonucleotide primer bearing the desired mutated sequence is synthetically prepared. This primer is then annealed with the single-stranded DNA preparation, taking into account the degree of mismatch when selecting hybridization (annealing) conditions, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected that include recombinant vectors bearing the mutated sequence arrangement.

Of course, the above described approach for site-directed mutagenesis is not the only method of generating potentially useful mutant peptide species and as such is not meant to be limiting. The present invention also contemplates other methods of achieving mutagenesis such as for example, treating the recombinant vectors carrying the gene of interest mutagenic agents, such as hydroxylamine, to obtain sequence variants.

E. Protein Purification

It will be desirable to purify the peptides of the present invention. Protein purification techniques are well known to those of skill in the art. These techniques involve, at one level, the crude fractionation of the cellular milieu to polypeptide and non-polypeptide fractions. Having separated the peptides or polypeptides of the invention from other proteins, the polypeptide or peptides of interest may be further purified using chromatographic and electrophoretic techniques to achieve partial or complete purification (or purification to homogeneity). Analytical methods particularly suited to the preparation of a pure peptide include size-exclusion chromatography, ion-exchange chromatography, hydrophobic interaction chromatography, isoelectric focusing and capillary electrophoresis. A particularly efficient method of purifying peptides is fast protein liquid chromatography (FPLC) or even high performance liquid chromatography (HPLC).

Certain aspects of the present invention concern the purification, and in particular embodiments, the substantial purification, of an encoded polypeptide, protein or peptide. The term “purified polypeptide, protein or peptide” as used herein, is intended to refer to a composition, isolated from other components, wherein the polypeptide, protein or peptide is purified to any degree relative to its naturally-obtainable state. A purified polypeptide, protein or peptide therefore also refers to a polypeptide, protein or peptide, free from the cellular environment in which it may naturally occur.

Generally, “purified” will refer to a polypeptide, protein or peptide composition that has been subjected to fractionation to remove various other components, and which composition substantially retains its expressed biological activity. Where the term “substantially purified” is used, this designation will refer to a composition in which the polypeptide, protein or peptide forms the major component of the composition, such as constituting about 50%, about 60%, about 70%, about 80%, about 90%, about 95% or more of the proteins in the composition.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified polypeptide, protein or peptide.

Assays for Determining the Efficacy of Treatment Protocols

As discussed herein throughout, compositions comprising IL-17 and agonists of IL-17 or an IL-17 receptor will be useful in treating endometriosis and other infertility related disorders in which TNFα is implicated. As discussed above, there are numerous preparations of IL-17 that are known to those of skill in the art. Such preparations may all be tested to determine the efficacy of those agents in producing a therapeutic effect on inhibiting cell adhesion, such as e.g., TNFα-induced cell adhesion. Any inhibition of such cell adhesion will be a useful indicator that such an agent will be useful in the treatment of endometriosis. Such a determination could be made using in vitro and/or in vivo models of endometriosis.

A. In Vitro Assays for Endometriosis

Endometrial cells in cell culture may be used to determine the effects of various compositions on the adhesion of these cells. The endometrial cells may be primary cells isolated from an animal using techniques known to those of skill in the art. For example, cells from an endometrial biopsy may be taken and established as a primary cell culture or may be grown in culture over a prolonged period of time to produce a cell line. Techniques for establishing and maintaining such animal cell culture are known to those of skill in the art see e.g., R. Ian Freshney/Culture of Animal Cells: A Manual of Basic Technique, 4th Edition, Publ. Wiley & Sons, 1998. The primary cells may be from any mammalian source including human, primate, bovine, ovine, porcine and the like.

To obtain primary cell culture from endometrial tissue from a test animal such as a cow, pig, mouse, rat etc., the reproductive tract is removed and under a laminar flow hood, sterile scissors are used to expose the endometrium. Using forceps the uterine endometrium is lifted and excised with scissors to remove it from the underlying myometrium. During collection, the strips of endometrium are placed into a 100 mm petri dish containing an appropriate buffer. Using a scalpel and forceps, the endometrium is cut into explants of about 2-3 mm³. The explants are washed by aspirating old medium, adding fresh medium and swirling gently. This step reduces contamination of medium with serum proteins that can interfere with treatments and analysis of conditioned medium. For maximum reduction in serum protein contamination, the explants are incubated in medium for 2 h before the medium is replaced with fresh medium. Primary cultures are prepared by adding the required amount of medium to a clean petri dish and incubating the cells at 37° C. on a rocking platform in an atmosphere of 50:45:5 N₂:O₂:CO₂ (v/v/v). This gas can be special-ordered or prepared by mixing equal volumes of 100% N₂ and a 90:10 O₂:CO₂ mixture.

In other embodiments, the cell culture may be set up using an endometrial cell line. Again the source of such a cell line may be any mammalian source. In certain exemplary embodiments, the cell line is a bovine endometrial (BEND) cell line. Cultures of established BEND cells are well known to those of skill in the art see e.g., Johnson et al. Endocrine 10:243 (1999); Perry et al. Mol. Endocrinol. 13: 1197 (1999); Austin et al. Endocrinology 140: 542 (1999). Various endometrial cell lines are commercially available from depositories such as the American Type Culture Collection (Bethesda, Md.). Such cell lines include, e.g., ATCC cell line CRL-1622 (human endometrial cell), ATCC cell line CRL-1671 (human endometrial cell), ATCC cell line CRL-2398 (bovine endometrial cell); ATCC cell line CRL-2674 (mink endometrial cell); ATCC cell line CRL-2675 (mink endometrial cell). Each of these cell lines are supplied with exemplary protocols for culturing of these cell lines in culture.

In certain embodiments, the Examples provided herein employ the BEND ATCC cell line CRL-2398. The cells are thawed and the culture is established using e.g., the protocol provided with the cell lines from ATCC. The cells are suspended at an appropriate density e.g., 2×10⁵ cells/ml growth medium. Approximately 20000 cells are places into each well of a 96-well culture plate and the cells are allowed to grow for 24 hours. After the 24 hour incubation period, cells have grown as a monolayer on the culture plate. The culture medium is changed to one where the serum is substituted with 0.1% BSA and the test protein and/or TNFa and cultured for 3 days. To these cells is added an aliquot (e.g., 50 μl of medium containing 0.04% (v/v) fibronectin coated fluorescent beads. After an incubation time of e.g., 60 minutes, the medium is aspirated and the total fluorescence of the plate is determined. This procedure is performed with cells that have been incubated with buffer alone, TNFα alone or a combination of TNFα with one or more of the test compounds being tested for activity as inhibitors of TNFα-induced fibronectin-coated bead binding to BEND cells. A comparison of the binding done in the presence and absence of TNFα will reveal the amount of binding induced by TNFα. A comparison of that value with the binding induced in the presence of both TNFα and the test inhibitory compound will identify those compounds that act as inhibitors of the binding. In this manner, it was determined that IL-17 is such an inhibitory compound.

While the above discussion describes the binding of fibronectin coated beads to BEND cells, it should be understood that binding to any fibronectin-coated solid support may be used. Likewise, the solid support may be coated with molecules other than fibronectin that are involved in cellular adhesion of endometrial or mesothelial cells.

B. In Vivo Assays for Endometriosis

Alternatively and/or in addition to the above mentioned in vitro assays, the effects of IL-17 related compositions and other agents administered to affect remission, reduction of size or number endometriotic foci or otherwise alleviate the symptoms of endometriosis may be tested in established experimental models of endometriosis. Such models are known to those of skill in the art (see e.g., U.S. Pat. No. 6,663,865; Jones, Acta Endocrinol. (Copenh.) 114, 379-382, 1987; Dudley et al., Am. J. Obstet. Gynecol. 167, 1774-1780, 1992; Sharpe et al., Prog. Clin. Biol. Res. 323, 449-58, 1990). In such models, a rat, e.g., a female Sprague-Dawley rat (250-275 g) is induced to undergo experimental endometriosis as follows: The rat is placed under anaesthesia, and an autologous fragment of endometrial tissue (1 cm in length) is resected from the right uterine horn and placed in PBS at 37° C. The uterine segment is opened by a longitudinal incision, and a 5×5 mm section is transplanted, without removing the myometrium, onto the inner surface of the abdominal wall using non-absorbable silk suture at four corners.

A few weeks after inductions of endometriosis (e.g., three weeks), the animals are examined by laparotomy (pre-treatment laparotomy) in order to evaluate the size and viability of the ectopic endometrial tissue. The surface area (length×width) is measured using a caliper and recorded. Animals in which the graft is viable are selected as animal models for determining the efficacy of the treatment. Typically, a recovery period is allowed after the laparotomy prior to initiation of treatment protocols. While the above procedure is outlined for a rat model, it may be readily adapted to other animals.

In the test treatment protocols, one may employ a control group that receives only saline; another group receives the therapeutic agent being tested for its effect of reducing endometriosis. In preferred embodiments, the agent is IL-17, an agonist of IL-17 or an agent that stimulates the endogenous production of IL-17. The treatment protocol may use the equivalent of 10 ng/kg to up to 100 mg/kg of mammal body weight or more per day, preferably about 1 μg/kg/day to 10 mg/kg/day, depending upon the route of administration and site or target at which the agent is to be delivered. At designated sacrifice times, the animals are anaesthetized and blood samples are collected from abdominal aorta to determine the presence circulating factors that are indicative of endometriosis (e.g., presence of antiendometrial autoantibodies, presence of hormones associated endometrial tissue and the like). Meanwhile, tissue biopsies from ovaries, uterosacral ligaments, pelvic peritoneum, rectovaginal septum, cervix, vagina, the fallopian tubes, intestine, bladder, pleura, lymph nodes and the pancreas are examined for the presence of endometriosis-like foci. Any decrease in the size or number of these foci at any of these sites as a result of administration of any of the compositions described herein will be indicative that such a composition will be therapeutically useful. Thus, the use of such models allows the skilled artisan to assess the use of IL-17 and related compositions in the treatment of endometriosis-related infertility.

C. Other Functional Assays

In addition to the above endometriosis related assays, one of skill may wish to employ any of a number of assays to determine whether a given IL-17 possesses IL-17-like activity. IL-17 is a cytokine and assays for monitoring cytokine activity are well known to those of skill in the art. In particular, it has previously been described that IL-17 is a proinflammatory cytokine. Therefore any assay that monitors such a proinflammatory effect of IL-17 could be used. Such assays are described in e.g., Fossiez et al., Int. Rev. Immunol., 16:541-551 1998; U.S. Pat. No. 6,569,645; Javanovic et al., J. Immunol. 160:3513-3521, 1998. The capability of inducing granulopoiesis is typical of IL-17. Such activity may be measured using the assays described in Fossiez et al., J. Exp. Med. 183:2593-2603, 1996.

In some examples, skin vascular permeability assays can be used to assess whether the IL-17 polypeptide or variant thereof is able to stimulate an immune response and induce inflammation by inducing mononuclear cell, eosinophil and PMN infiltration at the site of injection of the animal. In such assays, an animal is anesthetized with ketamine (75-80 mg/Kg) and 5 mg/Kg Xylazine intramuscularly (IM) and a sample of the test polypeptide or a conditioned media test sample is injected intradermally onto the backs of the test animals with 100 μL per injection site. Multiple, e.g., 10-30, injection sites may be used per animal. One mL of Evans blue dye (1% in physiologic buffered saline) is injected intracardially. Blemishes at the injection sites are then measured (mm diameter) at 1 hr, 6 hrs and 24 hrs post injection. Animals are sacrificed at 6 hrs after injection. Each skin injection site is biopsied and fixed in paraformaldehyde. The skins are then prepared for histopathalogic evaluation. Each site is evaluated for inflammatory cell infiltration into the skin. Sites with visible inflammatory cell inflammation are scored as positive. Inflammatory cells may be neutrophilic, eosinophilic, monocytic or lymphocytic.

The IL-17 receptor also is known to the skilled artisan and U.S. Pat. Nos. 6,096,305, 6,919,104; U.S. Pat. No. 6,100,235; U.S. Pat. No. 6,072,033 and U.S. Pat. No. 5,869,286 each incorporated herein by reference teach methods of making cells that express IL-17 receptors. Methods of transfecting cells e.g. mammalian cells, with expression constructs that encode receptors that are expressed on the surface of the cell are well known in the art. (See, for example, U.S. Pat. No. 5,053,337; U.S. Pat. No. 5,155,218; U.S. Pat. No. 5,360,735; U.S. Pat. No. 5,472,866; U.S. Pat. No. 5,476,782; U.S. Pat. No. 5,516,653; U.S. Pat. No. 5,545,549; U.S. Pat. No. 5,556,753; U.S. Pat. No. 5,595,880; U.S. Pat. No. 5,602,024; U.S. Pat. No. 5,639,652; U.S. Pat. No. 5,652,113; U.S. Pat. No. 5,661,024; U.S. Pat. No. 5,766,879; U.S. Pat. No. 5,786,155; and U.S. Pat. No. 5,786,157, the disclosures of which are hereby incorporated by reference in their entireties into this application.) IL-17 receptor containing cells, e.g., cells produced by such transfection may readily be used to test any IL-17 derivative or variant to determine whether that derivative or variant will have an IL-17 receptor binding activity, and/or function as an agonist of the receptor. Such determinations are routine in the art, see for example, U.S. Pat. Nos. 5,053,337; 5,155,218; 5,360,735; 5,472,866; 5,476,782; 5,516,653; 5,545,549; 5,556,753; 5,595,880; 5,602,024; 5,639,652; 5,652,113; 5,661,024; 5,766,879; 5,786,155; and 5,786,157 (each incorporated by reference in their entireties into this application.)

The term “agonist” is used to indicate any IL-17-derived protein, peptide or peptide mimetic of a IL-17-derived protein compound which increases the activity of any IL-17 receptor of the subject invention. Preferably, such an agent also will serve to inhibit TNFα-induced binding of endometrial cells to form endometriotic calli/foci.

Typically, the receptor binding activity is determined by preparing a cell or a membrane preparation of a cell transfected with, and expressing the receptor, or obtaining a cell or a membrane preparation from a cell known to express said receptor, with the IL-17-derived protein to be tested under conditions permitting receptor-ligand binding.

Patient Selection and Monitoring

In the therapeutic embodiments, it will be desirable to identify patients that need the therapeutic intervention and also to provide a prognostic indicator of the efficacy of the treatment regimens being administered. The patients that receive the treatments of the invention will be any patients that manifest the signs of an infertility-related disorder that is involves by TNFα-induced cellular adhesion. Typically, such patients may be female patients between the age range of 20 to 50 years and manifest one or more symptoms of endometriosis. For example, most commonly women with this syndrome experience excessive, heavy or prolonged menstrual bleeding and painful periods (dysmenorrhea). The dysmenorrheal pain includes backache, diarrhea, dizziness, headache and nausea. Also, extensive involvement of the uterine muscle can interfere with the normal contractility of the muscle which leads to excessive bleeding. In many cases, the menses is very dark. The amount of bleeding and cramps usually is associated with the degree of involvement and depth of penetration of the endometrial tissue into the uterine walls. Pain and bleeding are characteristics that may be monitored by a clinician in testing the degree of initial disease and the efficacy of the treatment being applied.

Adenomyosis, or “endometriosis of the uterus”, is a common form of endometriosis in which the endometrial cells penetrate deep within the uterine muscle (myometrium) at the back wall (posterior side) of the uterus. This leads to an enlarged, distended, hard uterus. The disease may be localized, with well defined borders, or diffuse having no limits or borders. When a localized disease is found this is called adenomyoma. These adenomyomas can be located at different levels of the uterine muscle and can penetrate into the uterine cavity and become submucosal tumors.

Detection of endometriosis may be by way of a pelvic examination, which in the case of adenomyosis may reveal an enlarged uterus that may be very firm and tender to the touch. Alternatively, a dilation and cauterization procedure may reveal endometrial tissue which can then be assessed histologically for the presence of fibroid masses or polyps. Magnetic resonance imaging (MRI) and vaginal ultrasound techniques also are used where the endometriosis is present as a discrete mass. Those of skill in the art typically diagnose endometriosis using a laparoscopic examination of the site to be examined and performing long needle biopsy by inserting the needle into site of the endometrial cell callus and taking a tissue sample for pathological testing (Wolf and Singh Obstet Gynecol Surv., 44:89-956, 8, 1989).

Endometriosis produces fibrosis, solid masses, cysts, liquid effusion and inflammatory reaction, it has been shown that all of these characteristics distort the ultrasound image of whichever organ (e.g., uterus, ovaries, bowel, lungs, kidneys, etc.) is being monitored. Such determinations reveal one or more of the following characteristics: more interphases surrounding the uterus and ovaries, higher echogenecity around the uterus and ovaries, lower contrast, diffuse hypoechoic areas, surrounded by strong echogenic tissue, uterus malposition: retroflexion, retroversion, retrocession, lateral displacement (alone or combined), small low-level echo areas within the myometrium (adenomyosis), hypoechoic or mixed pattern cysts, polycystic or microcystic ovaries, fixed uterus and ovaries (transvaginal sonography), cul-de-sac abnormal effusion, ovarian enlargement, hypoechoic ovaries and pelvic vascular congestion. Therefore, ultrasound may be performed to assess one or more of these parameters both determine whether a patient has endometriosis and to provide a prognosis of the therapeutic efficacy of the treatment being administered.

Computed tomography (CT) scans have been used to diagnose endometriosis particularly where the lesions are small. (Grassi et al., J Comput Tomogr., 9:157-159; 1985). Other symptoms and signs for endometriosis and methods of determining endometriosis are well known to those of skill in the art, see e.g., Brosens, Am. J. Obstet. Gynecol. 176:263-7, 1997; Chatterjee et al., Obstet. Gynecol., 56:81-84, 1980; Ural and Badway, Hospital Physician, 33:47-48, 1997. U.S. Pat. No. 6,645,725 describes an immunoassay based method for the detection of endometriosis that may be useful in identifying patients for treatment regimens. In addition, Mid-South Fertility Institute (Knoxville, Tenn.) provides a commercial antiendometrial antibody (AEA) assay used to test a patient's blood for the presence of autoantibodies against endometrial antigens. Presence of these antibodies is an indication that endometriosis exists whereas absence indicates that endometriosis does not exist

As with most patient selections for treatment of female reproductive disorder, prior to and during therapy, the patient is subjected to a thorough gynecologic examination and endocrinologic evaluation, including an assessment of pelvic anatomy. The presence of cancerous masses should be excluded and it should be ensured that the patient is not pregnant.

In addition, it is contemplated that the present treatment methods may result in increase in ovulation. The clinical manifestations of ovulation, other than pregnancy, may be obtained either through a direct or an indirect measure of progesterone production. Such indicia include: a rise in basal body temperature, increase in serum progesterone, menstruation following a shift in body temperature. In conjunction with the above indicators of progesterone production, sonographic visualization of the ovaries may be used to assist in determining if ovulation has occurred. Such monographic monitoring may include evaluating fluid in the cul-de sac, ovarian stigmata and the presence of collapsed follicles.

In addition, it is contemplated that the present treatment methods may result in increase in frequency and efficiency of implantation. As indicated herein, the IL-17 also will be used to increase the decidualization of endometrial stromal cells so that the endometrium is receptive to embryo implantation. Decidualization involves the transformation of endometrial stromal fibroblasts into decidual cells. This is the major change that occurs in the primate endometrium after conception. In this process, the uterine endometrial stromal cells differentiate to decidual cells following the establishment of pregnancy. Decidual cells play an important role in implantation and provide nutritional support for embryo. Decidual cells are also believed to produce factors that control trophoblast invasion and protect the embryo from maternal immune rejection. During the process of decidualization in the primate, fibroblast-like stromal cells change morphologically into polygonal cells and begin to express specific decidual proteins. This is manifested by the downregulation of a smooth muscle actin expression and the induction of insulin-like growth factor binding protein-1 (IGFBP-1). IGFBP-1 gene expression in decidualizing stromal fibroblasts requires the presence of both hormones and cAMP. This induction is associated with a concomitant decrease of a-smooth muscle actin expression in vivo and in vitro. Other changes in increased decidualization include e.g., increased IL-1b expression, activation COX-2 expression, which may in turn increase PGE2 which can increase intracellular cAMP via activation of the EP2 and EP4 receptor. Any of these changes can readily be monitored as a marker of increased decidualization. Increased superoxide dismutase expression also is a marker of increased decidualization. Prolactin production also is seen with increased decidualization. Another characteristic of decidualization is that the stromal fibroblasts are enlarged in comparison to the nonpregnant precursors. Decidualization is a well-established process and methods of monitoring the gene expression level and other morphological changes associated with decidualization are well known to those of skill in the art. (see e.g., Daly et al., Am J. Obstet. Gynecol., 145:672-678, 1983; Sugino et al., Mol. Human. Reprod. 6(2):178-184, 2000; Zoumakis et al., Mol. Human. Reprod., 6(4): 344-351, 2000). Such methods include e.g., determining immunofluorescence of cytokeratins, determining presence of prolactin using chemiluminescence, determining presence of PGE2 using radioimmunoassays, determining presence of interleukins and chemokines (e.g., IL-6, IL-8, IL-11, GM-CSF) using standard ELISA techniques (see e.g., Zoumakis et al., Mol. Human Reprod., 6(4): 344-351, 2000).

Methods of Identifying Additional Agents to Affect TNFa-Induced Binding of Endometrial Cells

The present invention also contemplates identifying additional agents that act as agonists of IL-17 in inhibiting the TNFα-induced binding of endometrial cells to structures that have features of stromal cells. As shown herein, IL-17 inhibits this activity. In order to do this, the inventors set up an assay using BEND cells and determined the binding of fibronectin-coated beads to BEND cells in the presence and absence of TNFα either alone or in combination with IL-17. The TNFα increased the binding of beads to BEND cells. This correlates with in vivo observations which have shown that the peritoneal fluid of patients suffering from endometriosis contains elevated levels of this and other macrophage secretory products (Fiers, FEBS Lett. 285, 199-212, 1991; Hornung et al., J. Clin. Endocrinol. Metab. 82, 1621-1628, 1997; Harada et al., Am. J. Obstet. Gynecol. 176, 593-597, 1997; Arici et al., Mol. Hum. Reprod. 2, 40-45, 1996; Arici, et al., Fertil. Steril. 67, 1065-1072, 1997). The present invention shows a surprising finding that IL-17 inhibits the effects of TNFα on the cell adhesion properties of endometrial cells and can therefore be used as a therapeutic agent to treat endometriosis. This finding may further be exploited to identify additional agents that augment, enhance, or otherwise stimulate this effect of IL-17. The present section describes screening assays for identifying such compounds. In the screening assays of the present invention, the candidate substance may first be screened for basic biochemical activity—e.g., in vitro binding to an IL-17 receptor, or for increasing that binding property of IL-17 etc. The agent may then be analyzed in the BEND endometriosis assays described herein for effects on inhibiting the TNFα-induced binding of BEND cells to solid supports and subsequently be tested for its ability to decrease or alleviate the symptoms of endometriosis either through observations at the cellular, tissue or whole animal level. To this effect, animal models of endometriosis are known and have been discussed elsewhere herein.

The present invention provides methods of screening for agents that augment the inhibitory effect of IL-17 on TNFα activity in endometrial tissue. It is contemplated that such screening techniques will prove useful in the identification of compounds that will inhibit, or decrease the cell adhesion properties of endometrial cells and will thus be useful in the treatment of endometriosis. Further, as such compounds will likely remove or decrease the degree of endometrial-loci in the reproductive and other areas of an animal, such compositions will be useful in protocols for treatment of infertility. In these embodiment, the present invention is directed to a method for determining the ability of a candidate substance to modulate the effects of IL-17 on TNFα-induced binding of endometrial cells to a solid support, generally including the steps of:

i) determining the binding of endometrial cells in culture with a solid support in the presence of TNFα;

ii) determining the binding of endometrial cells in culture with a solid support in the presence of TNFα and IL-17; and P iii) comparing the binding in step (i) with the binding in step (ii).

This initial assay reveals the inhibitory effects of IL-17 on TNFα-induced endometrial cell binding. Performing step (iii) in the presence and absence of a candidate modulator of IL-17 will identify agents that further augment this inhibition. Any agent that further inhibits the TNFα-induced endometrial cell binding in excess of IL-17 will be a useful agent as a candidate therapeutic agent that can be tested in vivo models, such as those described herein.

To identify a candidate substance as being capable of modulating the effects of IL-17 in the assay above, one measures or determines the binding in the absence of the added candidate substance. One then adds the candidate substance to the cell or in a parallel assay and determines the response in the presence of the candidate substance. A candidate substance which further inhibits endometrial cell binding as compared to the assay performed with IL-17 also will be useful. In the in vivo screening assays of the present invention, the compound is administered to a model animal, over period of time and in various dosages, and an alleviation of the signs and symptoms associated with endometriosis are monitored. Any improvement in one or more of these signs or symptoms will be indicative of the candidate substance being a useful agonist of IL-17 activity.

As used herein the term “candidate substance” refers to any molecule that may potentially act as an agonist of IL-17. In certain aspects, the candidate substance is a protein or fragment thereof, a small molecule stimulator, or even a nucleic acid molecule. Alternatively, useful pharmacological compounds will be compounds that are structurally related to other known agonists of IL-17 or even antagonists of TNFα. Of particular interest in this latter regard is U.S. Pat. No. 6,663,865, which teaches various antagonists of TNF that may be useful in various combination therapies herein. Rational drug design includes not only comparisons with known modulators of IL-17, its receptor, and the like, but predictions relating to the structure of target molecules.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds molded of active, but otherwise undesirable compounds. Given the identification of the BEND assay provided herein, those of skill in the art will readily be able to conduct high throughput screening of such libraries.

Candidate compounds may include fragments or parts of naturally-occurring compounds or are found as active combinations of known compounds which are otherwise inactive. It is proposed that compounds isolated from natural sources, such as animals, bacteria, fingi, plant sources, including leaves and bark, and marine samples are assayed as candidates for the presence of potentially useful pharmaceutical agents. It will be understood that the pharmaceutical agents to be screened could also be derived or synthesized from chemical compositions or man-made compounds. Thus, the candidate substance identified by the present invention may be a polypeptide, a polynucleotide, a small molecule inhibitor or any other compound that is designed through rational drug design starting from a known activator of a IL-17 activity.

“Effective amounts” in certain circumstances are those amounts effective to reproducibly increase in the effects of IL-17. The candidate agent may even by one which increases the expression of IL-17 in endometrial or other reproductive tissue in comparison to the level of such expression in the absence of such compounds. Compounds that achieve significant appropriate changes in activity and/or expression of IL-17 will be used.

Significant changes in activity and/or expression will be those that are represented by alterations in activity of at least about 30%-40%, and in some aspects, by changes of at least about 50%, with higher values of course being possible.

In additional assays, the candidate substance is a variant or derivative of IL-17 and the derivative has a greater inhibitory effect than a wild-type IL-17.

Pharmaceutical Compositions

Pharmaceutical compositions for administration according to the present invention can comprise at least one IL-17-derived protein (e.g., a peptide of SEQ ID NO:2, 3, 4, or 5 a variant or analog thereof or any other IL-1,7-derived protein that inhibits the action of TNFα on endometrial cells and/or causes a decrease in endometrial callus mass formation).

The pharmaceutical compositions also may include other therapeutic agents. For example, while the present invention stems from the unique discovery of the therapeutic efficacy of IL-17 compositions in the treatment of infertility, it is contemplated that the IL-17 may be administered not only alone but in combination with other therapeutic regimens designed for the treatment of infertility in general as well as those treatments designed for the treatment of endometriosis in particular. As discussed above, endometriosis is generally treated with GnRH agonists (e.g., nafarelin acetate, leuprolide acetate, goserelin acetate, and buserelin acetate), with hormonal therapies including high dose oral contraceptive pills to mimic pseudopregnancy (using high doses of estrogen and progesterone), and Danazol. In addition, U.S. Pat. No. 6,663,865 describes the use of anti-TNF antibodies and antide for the treatment of endometriosis. Any such compositions may be used in combination with IL-17 to produce a combined therapy for the treatment of endometriosis and related infertility disorders. In addition, one or more of these treatments in combination IL-17-based therapy, or the IL-17 based therapy alone may be combined with surgical intervention. In addition, the IL-17 based therapies of the invention may be combined with any agent that is used for ovulation induction or other therapeutic intervention used in ART e.g., stimulation of gonadotropin release e.g., with clomiphene and letrozole, as well as various gonadotropins to increase ovulation induction and/or follicle maturation. Such gonadotropins would include e.g., FSH, and/or LH.

Each of the therapeutic preparations is preferably provided in a pharmaceutically acceptable form optionally combined with a pharmaceutically acceptable carrier. These compositions can be administered by any means that achieve their intended purposes. Individualized amounts and regimens for the administration of the compositions for the treatment of infertility using the methods of the present invention can be determined readily by those with ordinary skill in the art using the guidance provided herein to determine the efficacy of a dosage in an animal model and then to increase the dosage to higher mammals. Administration of Danazol, contraceptive hormones, GnRH agonists and other fertility related agents such as Serophene™, Clomid™, Fertinex™, Gonal F™, Bravelle™ and the like are known to those of skill in the art and can readily be found in the Physician's. Desk Reference. That document provides exemplary guidance as to types of formulations, routes of administration and treatment regimens that may be used in administering FSH. Any to of the protocols, formulations, routes of administration and the like described therein can readily be modified for use in the present invention.

Compositions within the scope of this invention include all compositions comprising at least one IL-17 formulated in an amount effective to achieve its intended purpose of inhibiting the action of TNFα-induced binding of endometrial cells and essentially acting as a TNFα antagonist. The active agents used in the methods of the present invention may be administered by any means normally employed for such administration. Most preferably, the IL-17-derived protein compositions used in the present invention are administered via injection, as a lotion, gel or salve or orally.

It is understood that the suitable dose of a composition according to the present invention will depend upon the age, health and weight of the recipient, kind of concurrent treatment, if any, frequency of treatment, and the nature of the effect desired. However, the most preferred dosage can be tailored to the individual subject, as is understood and determinable by one of skill in the art, without undue experimentation. This typically involves adjustment of a standard dose, e.g., reduction of the dose if the patient has a low body weight.

The total dose required for each treatment may be administered in multiple doses or in a single dose. The compositions may be administered alone or in conjunction with other therapeutics directed to the disease or directed to other symptoms thereof.

The compositions of the invention should be formulated into suitable pharmaceutical compositions, i.e., in a form appropriate for in vivo applications in a the therapeutic intervention of infertility disorders. Generally, this will entail preparing compositions that are essentially free of pyrogens, as well as other impurities that could be harmful to humans or animals, preferably for oral administration. The hormone formulations may be formulated akin to the currently available hormonal preparations. The peptide/protein formulations may be formulated similarly to any other small protein composition. Preferably, these formulations are for oral administration, however, other routes of administration are contemplated (e.g. injection, intrathecal administration and the like). As the reproductive organs are a significant site of endometriosis, it is contemplated that salves or lotions for delivery to the reproductive organs will be useful. Transdermal patches also may be used.

One will generally desire to employ appropriate salts and buffers to render the compositions stable and allow for uptake of the compositions at the target site. Generally the protein compositions of the invention are provided in lyophilized form to be reconstituted prior to administration. Buffers and solutions for the reconstitution of the compositions may be provided along with the pharmaceutical formulation to produce aqueous compositions of the present invention for administration. Such aqueous compositions will comprise an effective amount of each of the therapeutic agents being used, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the therapeutic compositions, its use in therapeutic compositions is contemplated. Supplementary active ingredients also can be incorporated into the compositions.

Methods of formulating proteins and peptides for therapeutic administration also are known to those of skill in the art. Administration of these compositions according to the present invention will be via any common route so long as the target tissue is available via that route. Most commonly, these compositions are formulated for oral administration. However, other conventional routes of administration, e.g., by subcutaneous, intravenous, intradermal, intramuscular, intramammary, intraperitoneal, intrathecal, intraocular, retrobulbar, intrapulmonary (e.g., term release), aerosol, sublingual, nasal, anal, vaginal, or transdermal delivery, or by surgical implantation at a particular site also may be used particularly when oral administration is problematic. The treatment may consist of a single dose or a plurality of doses over a period of time.

The active compounds may be prepared for administration as solutions of free base or pharmacologically acceptable salts in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients also can be incorporated into the compositions.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups also can be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration.

“Unit dose” is defined as a discrete amount of a therapeutic composition dispersed in a suitable carrier. Parenteral administration of the therapeutic compounds may be carried out with an initial bolus followed by continuous infusion to maintain therapeutic circulating levels of drug product. Those of ordinary skill in the art will readily optimize effective dosages and administration regimens as determined by good medical practice and the clinical condition of the individual patient.

The frequency of dosing will depend on the pharmacokinetic parameters of the agents and the routes of administration. The optimal pharmaceutical formulation will be determined by one of skill in the art depending on the route of administration and the desired dosage. Such formulations may influence the physical state, stability, rate of in vivo release and rate of in vivo clearance of the administered agents. Depending on the route of administration, a suitable dose may be calculated according to body weight, body surface areas or organ size. Further refinement of the calculations necessary to determine the appropriate treatment dose is routinely made by those of ordinary skill in the art without undue experimentation, especially in light of the dosage information and assays disclosed herein as well as the pharmacokinetic data observed in animals or human clinical trials.

Appropriate dosages may be ascertained through the use of established assays for determining blood levels in conjunction with relevant dose response data. The final dosage regimen will be determined by the attending physician, considering factors which modify the action of drugs, e.g., the drug's specific activity, severity of the damage and the responsiveness of the patient, the age, condition, body weight, sex and diet of the patient, the severity of any infection, time of administration and other clinical factors. As studies are conducted, further information will emerge regarding appropriate dosage levels and duration of treatment for specific diseases and conditions.

It will be appreciated that the pharmaceutical compositions and treatment methods of the invention may be useful in fields of human medicine and veterinary medicine. Thus the subject to be treated may be a mammal, preferably human or other animal. For veterinary purposes, subjects include for example, farm animals including cows, sheep, pigs, horses and goats, companion animals such as dogs and cats, exotic and/or zoo animals, laboratory animals including mice rats, rabbits, guinea pigs and hamsters; and poultry such as chickens, turkeys, ducks and geese.

The present invention also contemplated kits for use in the treatment of fertility disorders. Such kits include at least a first composition comprising the IL-17 based proteins/peptides described above in a pharmaceutically acceptable carrier. Another component may be an agent used in ART (e.g., FSH/LH etc.) in a pharmaceutically acceptable carrier. The kits may additionally comprise solutions or buffers for effecting the delivery of the first and second compositions. The kits may further comprise additional compositions which contain agents such as Danazol, OCPs and the like. The kits may further comprise catheters, syringes or other delivering devices for the delivery of one or more of the compositions used in the methods of the invention. The kits may further comprise instructions containing administration protocols for the therapeutic regimens.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Exemplary BEND Assay

The following example provides exemplary descriptions of a BEND assay for endometriosis used herein. Those of skill in the art will understand that culture conditions as well as the source of endometrial cells used in this assay may be changed but still produce the beneficial results of the assay.

All culture and assay plates were maintained in incubators with humidified chambers at 37° C. under 5% CO₂ and 95% air. Bovine endometrial cells (BEND cells ATCC CRL-2398) were used in the present examples merely because they are a readily available normal endometrial cell line. The BEND cell cultures were initiate d from frozen vials (each containing 2×106 cells/ml) that were rapidly thawed in a 37° C. water bath with continuous gentle agitation. The contents of each vial were transferred to a 75 cm² flask (Falcon cat# 353024) containing 10 ml supplemented-DMEM/F12. This medium consisted of DMEM/F12 medium (Gibco BRL cat# 11320033) containing 10% Horse Serum (HyClone cat# SH30074.03), 10% certified Fetal Bovine Serum (cFBS; HyClone cat# SH30071.3) and 25 U/ml each Penicillin and Streptomycin (Gibco BRL cat# 15070063).

After overnight incubation, the medium was replaced with supplemented-DMEM/F12 and the flasks were returned to the incubator. The cultures of BEND cells were split 1:3 or 1:4 when the monolayer was 100% confluent using a Trypsin-Versene mixture (Gibco BRL cat# 25300054). Briefly, the monolayer was washed with 15 ml of PBS (Gibco cat# 14190144) followed by addition of 5 ml of Trypsin-Versene. After the cells of the monolayer began to round up, the flask was tapped to further dislodge the monolayer and 15 ml of supplemented-DMEM/F12 was added. The medium with cells was then transferred to a 50 ml conical tube (Falcon cat# 352074) and the cells were pelletted at 200×g for 5 minutes. Cells were resuspended and plated for either culture maintenance or for the endometriosis assay.

The endometriosis assay was performed using BEND cells within 20 passages after receipt. The cells, after being trypsinized, were resuspended in 10 ml of supplemented-DMEM/F12 and transferred to a 15 ml conical tube (Falcon cat# 352095). 20 ul of cell suspension were used for the determination of cell number using a haemocytometer. After adjusting the cell density to 2×10⁵ cells/ml with supplemented-DMEM/F12 medium, an aliquot of the cell suspension (100 μl, 2000 cells/well) was added to each well of flat-bottom, 96-well black plates (Costar cat# 3603). After 24 hr the medium was aspirated and the monolayers rinsed once with 100 μl DMEM/F12 supplemented with 0.1% BSA (Sigma Chemical Co., St. Louis Mo. cat# A2058).

Fibronectin-coated beads were prepared in 20 ml glass scintillation vials (Fisher cat# 03-337-14) by coupling 1 ml (2% v/v solution) 0.5μ carboxylated polystyrene beads (Molecular Probes cat# F8813) with 3 mg of polymerized fibronectin (Sigma cat# F8141) dissolved in 300 μl H₂O and 200 μL of 100 mM MES (2-Morpholinoethanesulfonic acid Sigma cat# M3171) pH 6.8. Coupling was initiated with 100 μl of 100 mg/ml EDAC (Sigma cat# E7750) and allowed to proceed at room temperature for 2 hr with constant shaking. The beads were transferred to a 50 ml conical tube and washed in 45 ml of PBS by centrifugation at 2500×g for 15 min. The pellet was resuspended in 5 ml of 100 mM glycine (Sigma cat# G7403) and incubated for 30 minutes at room temperature. The beads were washed with 45 ml of PBS by centrifugation at 2500×g for 15 minutes. The pellet was resuspended in 5 ml of PBS.

For the assay, 100 μl of fresh DMEM/F12+0.1% BSA containing TNFα 15 ng/ml (R&D Systems cat# 210-TA) alone or with a dilution of test protein was added to each well. After a three day incubation, 50 μl of medium containing 0.04% (v/v) fibronectin-coated beads were added to each well. After 60 min incubation the medium was aspirated. Each well was then washed 3× with 100 μl PBS. A final 100 μl PBS was added and the plates were read for total fluorescence (490 nm excitation/515 nm emission) using an Analyst HT (LJL Biosystems, Sunnyvale, Calif.). The data were analysed using Microsoft Excel software and Origin software (version 7, OriginLab Corporation).

Test proteins were screened at three dilutions (1:50, 1:500, 1:5000), each in duplicate. Proteins that inhibited the action of TNFα on bead binding were considered positive and confirmed in a subsequent assay. Confirmed positives were tested in quadruplicate wells at serial dilutions of the test protein for dose-responsive activity. Every experiment was run with a negative control of DMEM/F12+BSA, a 15 ng/ml TNFα control, and a positive control of TNFα 15 ng/ml)+10 μg/ml TBP.

### Example 2

### IL-17 Inhibits TNFα-Induced Binding of Endometrial Cells to Support Cells

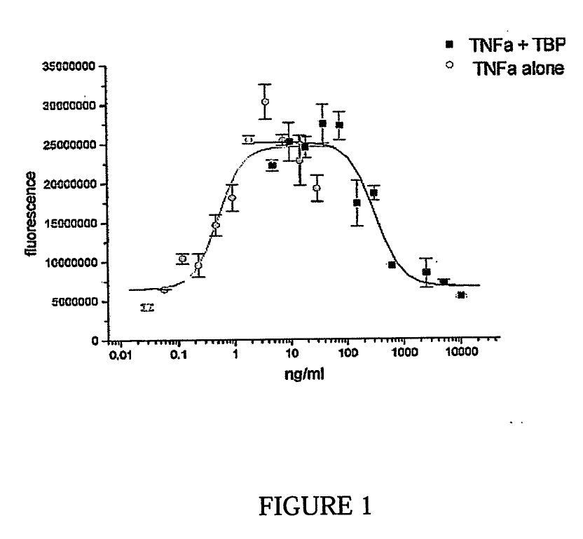

The bend assay was used to determine the effects of IL-17 on endometrial cells. Using an assay such as that described in Example 1, it was shown that TNFα increased binding of fibronectin-coated beads to BEND cells in a dose-dependent manner (FIG. 1). Addition of TBP during treatment with TNFα (15 ng/ml) reduced the bead binding to BEND cells in a dose-dependent manner (FIG. 1).

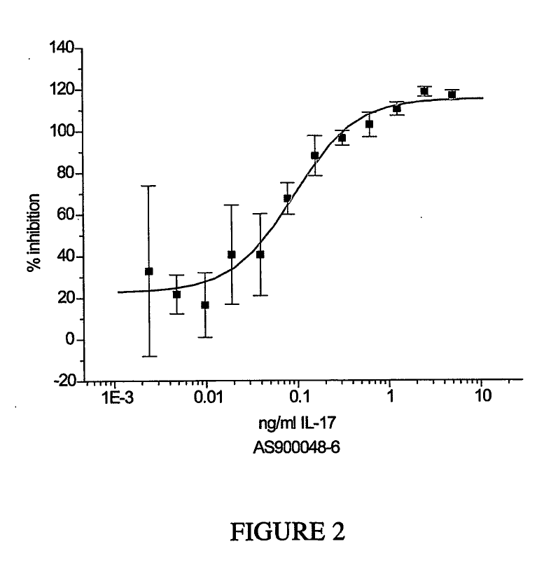

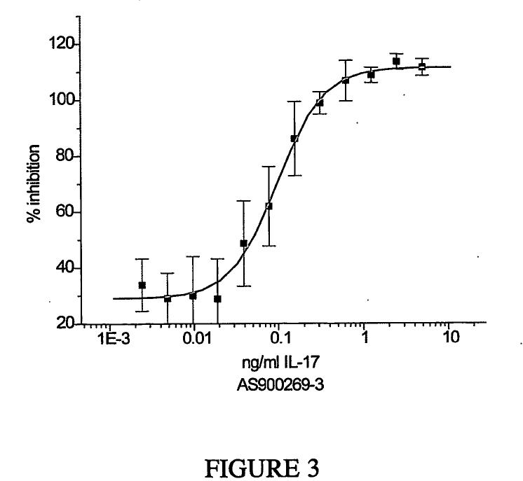

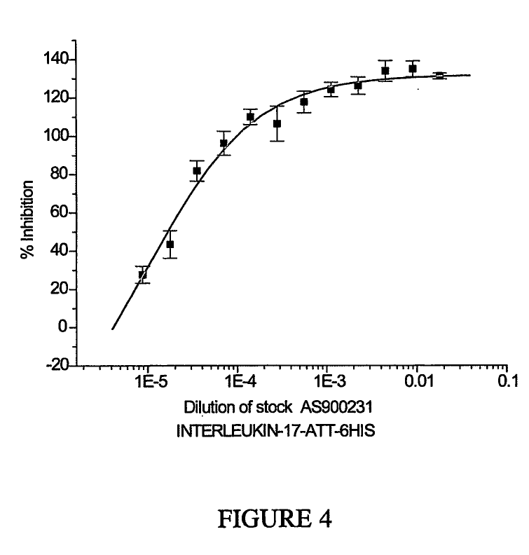

In further studies, Interleukin (IL)-17 GITIPRNPGC PNSEDKNFPR TVMVNLNIHN RNTNTNPKRS SDYYNRSTSP WNLHRNEDPE PNSEDKNFPR CRHLGCINAD GNVDYHMNSV PIQQEILVLR REPPHCPNSF RLEKILVSVG CTCVTPIVHH VAHHHHHH (SEQ ID NO:3) was identified as a positive protein in the inhibition of TNFα-induced binding of fibronectin-coated beads to BEND cells. After IL-17 was confirmed as a positive in this assay, it was tested for dose-responsive effects. Three different preparations of IL-17 were tested: IL-17-6HIS GITIPRNPGC PNSEDKNFPR TVMVNLNIHN RNTNTNPKRS SDYYNRSTSP WNLHRNEDPE RYPSVIWEAK CRHLGCINAD GNVDYHMNSV PIQQEILVLR REPPHCPNSF RLEKILVSVG CTCVTPIVHH VAHHHHHH (SEQ ID NO:3; FIG. 2), Met-IVKA IL-17 MIVKAGITIP RNPGCPNSED KNFPRTVMVN LNIHNRNTNT NPKRSSDYYN RSTSPWNLHR NEDPERYPSV IWEAKCRHLG CINADGNVDY HMNSVPIQQE ILVLRREPPH CPNSFRLEKI LVSVGCTCVT PIVHHVA (SEQ ID NO:4; originally procured through Peprotech cat# 200-17; FIG. 3) and IL-17 ATT-6HIS; GITIPRNPGC PNSEDKNFPR TVMVNLNIHN RNTNTNPKRS SDYYNRSTSP WNLHRNEDPE RYPSVIWEAK CRHLGCINAD GNVDYHMNSV PIQQEILVLR REPPHCPNSF RLEKILVSVG CTCVTPIVHH VANPAFLYKV VDIHHHHHH (SEQ ID NO:5; FIG. 4).

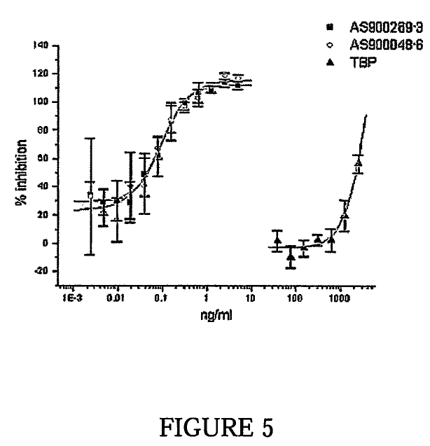

Within a single experiment Interleukin-17 and Met-IVKA IL-17 had similar efficacy in reducing binding of fibronectin-coated beads to TNFα-treated BEND cells (FIG. 5) and showed four-log greater efficacy in inhibiting the binding as compared to the positive control TBP. The third preparation (IL-17 ATT-6HIS) was tested as a dilution of a stock solution of unknown concentration (FIG. 4). All preparations of IL-17 inhibited the effect of TNFα on fibronectin-coated bead binding in a dose-dependent manner. The table below provides the IC₅₀ concentration of protein that inhibited TNFα-induced fibronectin-bead binding to BEND cells.

TABLE
Average IC₅₀ of two forms of IL-17 and TBP on TNFα-induced
binding of Fibronectin-coated beads to BEND cells

| Protein | # of experiments | IC50 X̅ ± std dev) |
|---|---|---|
| Met-IVKA | 5 experiments | 1.043 ± 1.35 ng/ml |
| IL-17 | | |
| IL-17-6HIS | 2 experiments | 0.181 ± 0.127 ng/ml |
| TBP | 4 experiments | 769.21 ± 319.44 ng/ml |

From the above data it is shown that IL-17 is effective at inhibiting TNFα-induced fibronectin-bead binding to BEND cells. These data demonstrate that IL-17 will have therapeutic efficacy against endometriosis because it is shown to decrease the binding of endometrial cells to agents that are normally found on cells to which endometrial cells adhere.

### Example 3

### Efficacy of IL-17 in Increasing Decidualization of Endometrial Stromal Cells

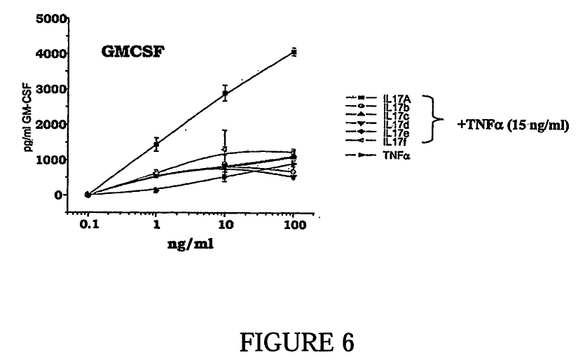

The above examples demonstrate the IL-17 is effective against endometriotic phenotypes. It is further demonstrated herein that IL-17 can be used to increase decidualization of endometrial stromal cells so that the phenotype of such cells is more akin to endometrial cells that are amenable, to embryo implantation. In the first set of experiments, IL-17 was evaluated for effects on cytokine production from 12Z endometriotic cells. Results shown in FIG. 6 demonstrate that addition of TNF-α to cultures of 12Z endometriotic epithelial cells had minimal effect on production of GM-CSF. Addition of IL-17A-F isoforms also had minimal impact on GM-CSF production with the exception of L-17A isoform. Combination of IL-17A and TNF-a caused a synergistic elevation in GM-CSF production. These results indicate that the effect of IL-17A is to increase both the sensitivity of cells to TNF, and also to increase maximum GM-CSF production from 12Z cells.

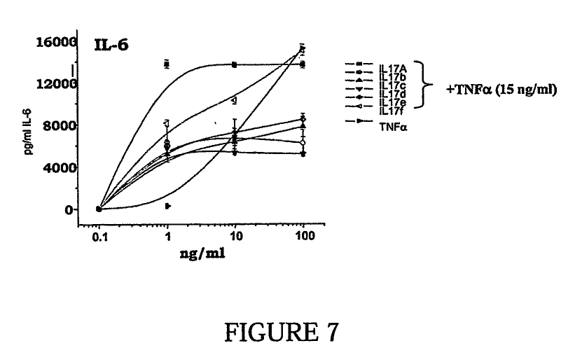

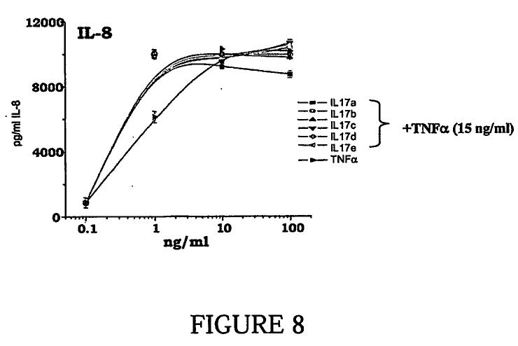

Results shown in FIGS. 7 and 8 illustrate that maximal production of IL-6 and IL-8 can be achieved with TNF-α. In FIG. 7, addition of TNF-a to cell cultures of 12Z endometriotic epithelial cells causes an increase in IL-6 production at concentrations above 1 ng/ml. In FIG. 7 the effect of IL-17 isoforms is to increase the sensitivity of epithelial cells to TNF-a without an additional increase in maximal IL-6 production with this combination. The sensitizing effect of IL-17 was observed for IL-17A and IL-17F isoforms, and there was no detectable effect for IL-17B-E isoforms. These results indicate that TNF-α causes a significant elevation in IL-6 production by itself, and the effect of IL-17 is to increase sensitivity of cells to the effect of TNF-α.

In FIG. 8, addition of TNF-α to cell cultures of 12Z endometriotic epithelial cells causes a significant increase in IL-8 production at concentrations as low as 1 ng/ml. There is no detectable effect of IL-17 isoforms on IL-8 production with this combination.

Results from experiments conducted with endometriotic epithelial cells demonstrate a clear effect of IL-17 to synergize with TNF-a in the production of GM-CSF production. The increase in sensitivity and maximum response in GM-CSF production distinguish this response compared to that of IL-6 and IL-8 production. Previously a role for GM-CSF has been established for paracrine modulation of stromal cells and influence the decidualization process during the window of implantation. Therefore, the present data provide evidence that first that IL-17 may reverse the transdifferentiation of endometriotic or adenomyotic epithelial cells back to a normal endometrial cell that could undergo decidualization. In the case of adenomyosis, stimulation of a decidualization process could facilitate a normal process of differentiation, and may be followed by elimination of these cells during a menses. In the case of endometriosis, a similar reversion of the cells is thought to occur, but due to their inappropriate location in the peritoneal cavity the reversion caused by GM-CSF may facilitate immune recognition and elimination of cells contributing to a lesion and reduce the severity of disease.

In stromal cells, both pro-inflammatory cytokines, TNF-α and IL-1β have been shown to be facilitate morphological decidualization of endometrium through production of biochemical mediators such as cytokines. Decidualization results in a differentiated cell population that produces IL-6, IL-8, IL-11, prolactin, and IGF-BP-1. The effects of IL-17 and TNF-α, as well as IL-1βwere examined in HuF6 cells. These cells are human endometrial stromal cells that have previously been demonstrated to be a useful model for the study of morphological and biochemical mechanisms surrounding decidualization and implantation (Fazleabas). The results presented below demonstrate a synergistic effect of IL-17 and TNF-α on production of GM-CSF, IL-6 and IL-8 production. The results with the combination of IL-17 and IL-1β but the results suggest that there is not a synergistic effect between IL-1β and IL-17 on the maximal production of GM-CSF, IL-6 or IL-8 cytokines. These results also illustrate a distinct potency and maximum response difference between TNF-α and IL-1β on biochemical decidualization, an observation not reported in previous literature. In fact this may be an important distinction if IL-1β represents the normal mediator of decidualization in an endometrium. Responses observed in the presence of TNF-α alone may represent response of endometrium that is compromised in its ability to undergo decidualization and implantation. The effects of IL-17, demonstrated in the figures herein, in stromal and epithelial cells may represent a mechanism to facilitate the normal process of decidualization in the midst of a compromised endometrium.

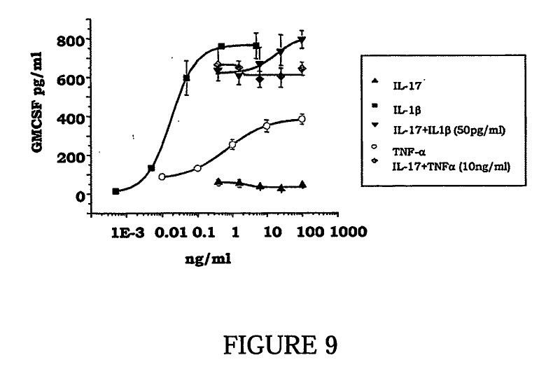

In FIG. 9, the combined effects of IL-17 and either TNF-α or IL-1β on GM-CSF production are shown. From this figure it is apparent that the concentration of IL-1β required for a maximum increase in GM-CSF production is significantly lower than the concentration of TNF-α. In addition, the maximum effect on GM-CSF production is achieved with IL-1β is greater than the maximum response with TNF-α. Addition of IL-17 to cultures also containing TNF-a increased the sensitivity of cells to TNF-α and increased the maximum amount of GM-CSF produced in the presence of TNF-α. Maximal levels of GM-CSF production response to IL-17 and TNF-α were similar to maximal levels obtained with either IL-1β or the combination of IL-1β and IL-17. Although the concentration response curve for effects of IL-17 is incomplete in this current set of preliminary results, the effects are discernible. The effects of IL-17 on IL-1β-stimulated GM-CSF production are less dramatic than with TNF-α. Addition of IL-17 in the presence of 50 pg/ml IL-1β stimulated a 20% increase in GM-CSF production that in this limited set of results seemed to reduce the sensitivity of cells to IL-1β.

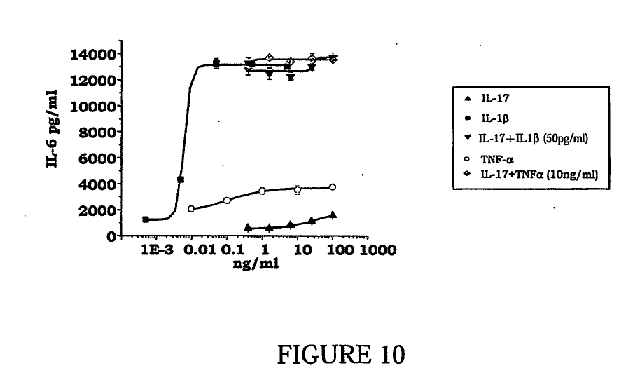

In FIG. 10, the combined effects of IL-17 and either TNF-a or IL-1b on IL-6 production are shown. From this figure it is again apparent that the concentration of IL-1β required for a maximum increase in IL-6 production is significantly lower than the dose of TNF-α. In addition, the maximum effect of IL-1β on IL-6 production is greater than the maximum response with TNF-α. Addition of IL-17 to cultures also 10 ng/ml TNF-a drastically increased the sensitivity of cells to TNF-α and markedly increased the maximum amount of IL-6 produced in the presence of TNF-α. Maximal levels of IL-6 production response to IL-17 and TNF-α were similar to maximal levels obtained with either IL-1b or the combination of IL-1b and IL-17. There were no detectable effects of IL-17 on IL-1β stimulated IL-6 production.

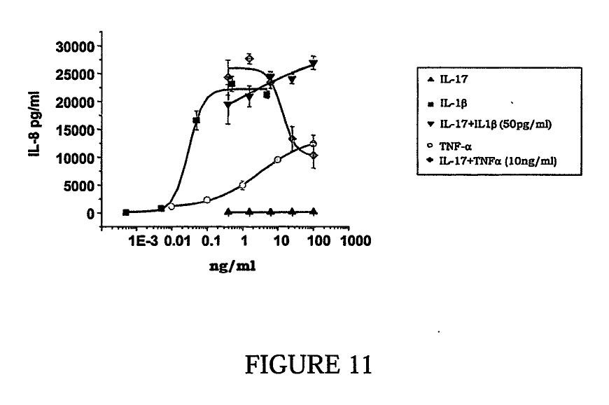

In FIG. 11, the combined effects of IL-17 and either TNF-α or IL-1β on IL-8 production are shown. From this figure it is once again apparent that the concentration of IL-1β required for a maximum increase in IL-8 production is significantly lower than the dose of TNF-α. In addition, it is consistently apparent that the maximum effect of IL-1β on IL-8 production is greater than the maximum response with TNF-α. Addition of IL-17 to cultures containing 10 ng/ml TNF-α appears to have increased the sensitivity of cells to TNF-a and certainly increased the maximum amount of IL-8 produced in the presence of TNF-α. Maximal levels of IL-8 production in response to IL-17 and TNF-α were similar to maximal levels obtained with either IL-1β or the combination of IL-1β and IL-17. There were no detectable effects of IL-17 on IL-1β-stimulated IL-8 production.

The results presented demonstrate an important interaction between TNF-α and IL-17. The current results suggest that IL-17 has significant ability to modify the response of cells to TNF-α. In the case of endometriosis, the data suggest that the effect of IL-17 or an antibody directed against TNF-α will prevent progression of the disease through modifying the cellular fate of endometrial epithelial and stromal cells. In the case of implantation, it is believed that IL-17 will facilitate decidualization in the presence of inflammatory cytokines that normally impede transformation of these cells for accepting embryonic implantation. Indeed as indicated above, it may be that IL-17 (as opposed to IL-1β) is able to facilitate decidualization in endometrium tissue that has been compromised and as such, IL-17 will be effective at facilitating implantation in endometrial tissues which have undergone endometriosis or have otherwise been comprised. In this manner IL-17 compositions will be useful as compositions for the treatment of infertility that results from aberrations in endometrium physiology, and particularly in mammals suffering from endometriosis.

### Example 4

### Treatment of Animals with IL-17

Those of skill in the art are aware of animal models for endometriosis (see e.g., studies of see e.g., U.S. Pat. No. 6,663,865; Jones, Acta Endocrinol. (Copenh.) 114, 379-382, 1987; Dudley et al., Am. J. Obstet. Gynecol. 167, 1774-1780, 1992; Sharpe et al., Prog. Clin. Biol. Res. 323, 449-58, 1990 discussed above). Such animal models are used to test the in vivo efficacy of the IL-17 compositions as therapeutic agents for endometriosis. Animals in these studies are divided into controls that do not receive any treatment and test animals which receive IL-17 based therapy.

Varying concentrations of IL-17 are administered to such animals as therapeutic compositions for the treatment of endometriosis. The doses of IL-17 may be varied according to the route of application. The route of application may be any route routinely used to administer cytokines.

The symptoms of endometriosis are monitored before and after treatment. Tissue samples (e.g., obtained by needle biopsy as described above) are evaluated before and after treatment for general appearance of endometrial tissues/vesicles therein. Additional procedures for determining the effects of the therapy include sonography.

A composition of the present invention is typically administered orally or parenterally in dosage unit formulations containing standard, well known non-toxic physiologically acceptable carriers, adjuvants, and vehicles as desired. The term parenteral as used herein includes subcutaneous injections, intravenous, intramuscular, intra-arterial injection, or infusion techniques. It is particularly contemplated that the therapy administered injection, oral or may be administered topically as a transdermal application (for example, by patch or vaginal cream), or by implant in an appropriate controlled release composition. The composition may be delivered to the animal alone or indeed in combination with any of the other therapies discussed herein throughout. A typical treatment course may comprise about multiple doses delivered daily or several times a day.

These animal models are used to identify the initial dosage ranges and protocols for the treatment of higher mammals, such as primates, and particularly humans. In such higher animals, the clinician may chose a regimen to be continued over a period of time, e.g., weeks or months.

Clinical responses in both the test animals and the human subjects being treated may be defined by any acceptable measure that is indicative of endometriosis. For example, a complete response may be defined by the disappearance of all measurable endometrial loci swelling for at least a month. Whereas a partial response may be defined by a 50% or greater reduction in the loci. Using the findings from the animal studies as a guideline, human trials can be performed using a starting dose selected as that dose where there is less than a grade 3 level toxicity. Dose escalation may be done by 100% increments until drug related grade 2 toxicity is detected and thereafter the dose escalation is stopped.

Of course, the above-described treatment regimes may be altered in accordance with the knowledge gained from clinical trials. Those of skill in the art will be able to take the information disclosed in this specification and optimize treatment regimes based on the clinical trials and animal studies.

Once such doses are identified, human trials may be initiated. In such trials a group of women presenting with the classic symptoms of endometriosis are enlisted from treatment. Symptoms and signs of endometriosis are monitored for up to twelve months. Pain scores are determined as follows. The patients evaluate their own dysmenorrhea, dyspareunia, and pain unrelated to menses on a scale of 0 to 3 (absent, mild, moderate, and severe). In addition, the investigators use the same scale for induration and tenderness. The patients' and investigators' ratings are combined to yield a profile of symptoms and signs: none (0), mild (1 to 2), moderate (3 to 5), severe (6 to 10), and very severe (11 to 15). Thus, the maximal score is 15. These numbers form an exemplary and arbitrary scale and other scales may be used as long as the study uses the same scale for all the participants that are compared to each other. Having determined the score in the absence of administration of the therapy, the therapy is initiated and the effects of the therapy are scored throughout the trial, at least on a monthly basis. Other indicators of relief of endometriosis also may be monitored using parameters such as ultrasound monitoring of endometrial loci, CT scans, needle biopsies of the tissue and the like. In this manner the amount and frequency of dosing regimens may be identified.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

The references cited herein throughout, to the extent that they provide exemplary procedural or other details supplementary to those set forth herein, are all specifically incorporated herein by reference.

What is claimed is:

1. A method for treating a fertility-related disorder in a mammal comprising administering to said mammal a composition comprising IL-17 in an amount effective to alleviate the symptoms of said fertility related disorder.

2. A method for treating endometriosis in a mammal comprising administering to said mammal a composition comprising IL-17 in an amount effective to alleviate at least one symptom of endometriosis in said mammal.

3. The method of claim 2, wherein said IL-17 is administered in an amount effective to inhibit the cellular adhesion of sloughed endometrial tissue to surrounding tissue.

4. The method of claim 3, wherein said administration of IL-17 reduces the number of endometrial lesions in said mammal.

5. The method of claim 1, further comprising administering an agent that inhibits the production, activity or expression of TNFα or its receptor.

6. The method of claim 5, wherein said agent is an anti-TNF antibody or a fragment thereof.

7. The method of claim 1 or claim 5, further comprising administering an agent that treats endometriosis.

8. The method of claim 1, wherein said composition comprising IL-17 is administered in combination with surgical resection of endometrial tissue.

9. The method of claim 8, wherein said composition comprising IL-17 is administered before, after or during said surgical resection.

10. The method of claim 2, further comprising administering hormone therapy.

11. The method of claim 10, wherein said hormone therapy comprises administering a gonadotropin releasing-hormone (GnRH) agonist.

12. The method of claim 11, wherein the GnRH agonist is selected from the group consisting of nafarelin acetate, leuprolide acetate, goserelin acetate, and buserelin acetate.

13. The method of claim 11, wherein said composition comprising IL-17 is administered concurrently with said GnRH agonist or is administered upon cessation of GnRH agonist.

14. The method of claim 11, further comprising administering to said individual an estrogen agent with a progestin agent to the woman on a daily basis for a period of time sufficient to prevent the symptoms of endometriosis.

15. The method of claim 14, wherein the estrogen agent is administered at a level that is biologically equivalent to about 5 to about 35 micrograms of ethinyl estradiol and the progestin agent is biologically equivalent to about 0.2 to about 1.5 milligrams of norethindrone acetate.

16. The method of claim 15, wherein the estrogen agent and the progestin agent are coadministered transdermally or orally.

17. The method of claim 16, wherein the oral administration of estrogen agent and the progestin agent is as a monophasic tablet or capsule.

18. A method for promoting decidualization of endometrial stromal cells in a mammal comprising administering to said mammal a composition comprising IL-17 in an amount effective to render the endometrial stromal cells of said mammal receptive to implantation as compared to cells of said mammal prior to administration of said composition.

19. The method of claim 18, wherein the promotion of decidualization of said cells is monitored using a characteristic selected from the group consisting of an increase IGFBP-1 expression, an increase in IL-1b expression, activation COX-2 expression, an increase in intracellular cAMP, decreased a-smooth muscle actin expression, an increase in PGE2 production, an increase in prolactin production, an increase in amount of oocyte implantation, and a swelling of endometrial stromal cells in response to said IL-17 administration.

20. The method of claim 18, wherein said mammal is suffering from endometriosis and administration of said IL-17 reverses the endometriotic phenotype of the endometrial cells to a phenotype that is receptive to embryo implantation.

21. A method of screening for an agent that inhibits TNFα-induced endometrial cell adhesion said method comprising:
i) determining the binding of endometrial cells in culture with a solid support in the presence of TNFα;
ii) determining the binding of endometrial cells in culture with a solid support in the presence of TNFα in the presence of IL-17;
iii) determining the binding of endometrial cells in culture with a solid support in the presence of TNFα, IL-17 and a candidate agent; and comparing the binding in step (ii) with step (iii), wherein a decrease in binding between step (ii) and step (iii) indicates that the agent is an inhibitor of TNFα-induced endometrial cell adhesion.

22. A composition comprising IL-17 for use in the treatment of infertility.

23. A composition comprising IL-17 for use in the treatment of endometriosis.
